(19) United States
(12) Patent Application Publication
(10) Pub. No.: US 2018/0272034 A1
(43) Pub. Date: Sep. 27, 2018

(54) USE OF CSA COMPOUNDS TO PREVENT MICROBIAL BUILD-UP OR FOULING OF MEDICAL IMPLANTS

(71) Applicants: Paul B. Savage, Mapleton, UT (US); Carl Genberg, Las Vegas, NV (US); Ronald L. Bracken, Monroe (GA)

(72) Inventors: Paul B. Savage, Mapleton, UT (US); Carl Genberg, Las Vegas, NV (US); Ronald L. Bracken, Monroe (GA)

(73) Assignee: BRIGHAM YOUNG UNIVERSITY, Provo, UT (US)

(21) Appl. No.: 15/926,577

(22) Filed: Mar. 20, 2018

Related U.S. Application Data
(60) Provisional application No. 62/474,499, filed on Mar. 21, 2017.

(51) Int. Cl. A61L 27/28; A61L 27/18; A61L 27/52; A61L 27/54; A61L 29/08; A61L 31/08; A01N 45/00

(52) U.S. Cl. CPC A61L 27/28; A61L 27/18; A61L 27/52; A61L 27/54; A61L 2300/606; A61L 31/08; A01N 45/00; A61L 2300/222; A61L 2300/404; A61L 29/08

(57) ABSTRACT

This disclosure describes the use of cationic steroidal antimicrobial (CSA) compounds to prevent microbial fouling of medical implants, including microbial fouling caused by bacterial and/or fungal biofilms. The CSAs are incorporated into the medical implants to provide effective antimicrobial properties. A medical implant includes a component formed from a polymeric material. A plurality of CSA molecules are mixed with the polymeric material so that the CSA molecules are incorporated into the structure of the medical implant as formed. A medical implant can additionally or alternatively include a lubricious coating containing CSA molecules.

### CROSS-REFERENCE TO RELATED APPLICATIONS

This Application claims the benefit of U.S. Provisional Patent Application No. 62/474,499, filed Mar. 21, 2017, the disclosure of which is incorporated herein by reference in its entirety.

### BACKGROUND

### 1. Field of Disclosure

The disclosure relates generally to methods of using one or more CSA compounds to prevent the fouling of medical implants due to microbial colonization and buildup.

### 2. Related Technology

Medical implants may be deployed onto and/or into a subject's body for diagnostic or therapeutic purposes. Medical implants may be intended either as a permanent or temporary implant. However, even when strict sterilization procedures are followed, medical implants can be subject to microbial contamination. In particular, formation of a biofilm on the medical implant can render the implant unfit for its intended use and/or even dangerous to the subject. When such fouling of the implant occurs, the implant must be removed from the subject, requiring additional medical treatment, including additional surgery, in many circumstances.

When a medical implant has fouled, the implant will be unfit for further use and must be discarded. Usually, however, the patient still requires the therapy the fouled implant was designed to treat, and the fouled implant must often be replaced with a new implant. This further adds to medical care costs, requiring the purchase of the new implant and associated medical costs of inserting the implant, and increases trauma to the patient during removal and replacement of the fouled implant.

Further, fouling of an implant is often associated with detrimental health effects. In many circumstances, an implant serves as a site for microbial contamination and biofilm formation, which may lead to recurrent and difficult to manage infections. These infections can occur at tissue sites near the implant, or can even occur at other remote locations in the subject's body. A microbial infection associated with a fouled implant can cause serious health problems for the patient, and can lead to serious, even deadly, conditions, such as sepsis. Even when treatable, these implant-associated infections require additional medical care, with its concomitant costs, prolonged healing times, and patient discomfort and trauma.

Particular concern exists over implant fouling caused by biofilms, including fouling caused by fungal biofilms or colonies. An increasing proportion of implantable medical device-related infections are being caused by Candida spp. As with other implant-related infections, management of implant-related Candida infections can be challenging and often requires removal of the infected implant. C. albicans biofilms have been reported to be 30 to 2,000 times more resistant to fluconazole, amphotericin B, flucytosine, itraconazole, and ketoconazole than planktonic cells. See Kojic and Darouiche, “Candida Infections of Medical Devices,” Clin. Microbial Rev. 2004 April 17(2): 255-267.

### BRIEF SUMMARY

Disclosed herein are methods for preventing microbial colonization and fouling of medical implants using one or more cationic steroidal antimicrobial (CSA) compounds. In some embodiments, a medical implant is treated with and/or manufactured to include a plurality of CSA molecules to provide the implant with anti-fouling properties.

Non-limiting examples of medical implants which may incorporate one or more CSA compounds, as described herein, include catheters, vascular catheters, peritoneal dialysis catheters, urinary catheters, joint prostheses, penile implants, dialysis access devices, dialysis access grafts, hemodialysis devices, fistula devices, hemodialysis grafts, cardiac devices, prosthetic valves, pacemakers (including implantable cardioverter defibrillators, or ICDs, and vascular assist devices, or VADs), central nervous system devices (VPSs), endotracheal tubes, intravenous (IV) needles, IV feed lines, other IV components, feeder tubes, drains, prosthesis components (e.g., voice prostheses), peristaltic pumps, tympanostomy tubes, tracheostomy tubes, oral care devices (dentures, dental implants), intrauterine devices (IUDs), cardiac implants, and dermal fillers. The medical implants described herein may be provided for use with a human or animal patient/subject. They may be for short-term or long-term implantation. Embodiments described herein may be particularly advantageous in applications where device biofouling, device rejection, and associated infection pathologies are common issues.

Additional examples of medical implants include medical devices which, in use, are implanted into a subject's tissues, deployed at a puncture or wound site, positioned for introducing or withdrawing material from a body cavity, or otherwise associated with a patient/subject in such a way that biological compatibility is of concern (e.g., because infection and/or fouling of the implant can result).

In some embodiments, a medical implant including CSA molecules provides antimicrobial properties and thereby provides the benefits of reducing fouling of the implant, reducing infection risk associated with fouling of the implant, reducing infection-related inflammation associated with the implant, reducing patient discomfort associated with an infection, and/or enabling more positive outcomes following a medical treatment involving a medical implant.

One or more embodiments are directed to methods of preventing microbial colonization and growth on a medical implant and likewise preventing infection at the implant site and the spread of microbial growth to other areas of a subject's body (e.g., when an infection becomes septic). One or more embodiments are directed to preventing biofilm formation on a medical implant. Beneficially, at least some of the embodiments described herein prevent microbial fouling of the medical implant caused by fungal and/or bacterial biofilms.

In some embodiments, a method of preventing microbial fouling of a medical implant includes (1) providing a medical implant having incorporated CSA molecules, as described herein, (2) implanting the medical implant, and (3) the medical implant incorporating the CSA molecules killing microbes contacting the medical implant or preventing adherence of microbes contacting the medical implant to thereby prevent microbial colonization of the medical implant. The medical implant may be effective in killing and/or disrupting adherence and colonization of a wide variety of microbes (e.g., a wide variety of different bacterial and/or fungal strains).

One or more embodiments are directed to methods of manufacturing a medical implant with incorporated CSA molecules to prevent or reduce microbial fouling of the implant. In some embodiments, such a method includes: (1) providing a medical implant; and (2) applying a coating to at least a portion of a surface of the medical implant to associate the coating with the medical implant, the coating being formulated to comprise CSA molecules.

In some embodiments, a method of manufacturing a medical implant with incorporated CSA molecules to prevent or reduce microbial fouling of the implant includes: (1) providing a biologically compatible moldable polymeric material; (2) mixing CSA molecules with the moldable polymeric material; and (3) molding the moldable polymeric material into a medical implant. In some embodiments, the CSA molecules are provided in salt form, such as a naphthalenedisulfonic acid (NDSA) salt, including 1,5-NDSA salt, of one or more CSA compounds. In some embodiments, molding the moldable polymeric material includes extruding the material through an extruder. In other embodiments, the medical device is formed using injection molding or other polymer molding or shaping process known in the art. By way of example, the CSA compound can be an NDSA salt of CSA-131 and/or similar CSA compounds.

The use of CSA compounds to prevent colonization of microbes and fouling of medical implants is unique in that CSAs can prevent both fungal and bacterial contamination. In general, the combination of fungal and bacteria growing on the same device leads to increased virulence and poor clinical outcomes, including higher rates of mortality. The CSA compounds provide a solution to this previously untreatable, or hard to treat, condition.

As used herein, the term “polymeric material” can refer to an already-formed polymer or to a polymerizable material or mixture that is capable of forming a polymer upon activation, curing, setting, etc. The polymeric material may be any polymer or polymerizable material suitable for medical use as part of a medical implant, including thermoplastic or thermoset materials. Some embodiments are directed to medical implants formed at least partly of silicone. Other medical implant embodiments may include polyethylene, polypropylene, polystyrene, polyester, polycarbonate, polyvinyl chloride, polyacrylate, polysulfone, or combinations thereof.

Additional features and advantages will be set forth in part in the description that follows, and in part will be obvious from the description, or may be learned by practice of the embodiments disclosed herein. It is to be understood that both the foregoing brief summary and the following detailed description are exemplary and explanatory only and are not restrictive of the embodiments disclosed herein or as claimed.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIGS. 1A-1C illustrate example cationic steroidal antimicrobial compounds; and

FIG. 2 compares histological images of tracheal tissue of a pre-term lamb intubated with an endotracheal tube not coated with a CSA-containing coating (panel A) to tracheal tissue of a pre-term lamb intubated with an endotracheal tube coated with a CSA-coating (panel B).

### DETAILED DESCRIPTION

### I. Overview of CSA Compounds

Cationic steroidal antimicrobial (“CSA”) compounds (“CSAs”), which are also known as “ceragenin” compounds (or “ceragenins”), are synthetically produced small molecule chemical compounds that include a sterol backbone having various charged groups (e.g., amine, guanidine, and/or other groups capable of exhibiting cationic properties under biological conditions) attached to the backbone. The backbone can be used to orient the cationic groups on one face, or plane, of the sterol backbone. In general, the term “CSA compound” refers to the type or structure of the CSA, while the term “CSA molecule” refers to the CSAs themselves when used in a medical implant.

CSAs are cationic and amphiphilic, based upon the functional groups attached to the backbone. They are facially amphiphilic with a hydrophobic face and a polycationic face. Without wishing to be bound to any particular theory, it is theorized that the CSA compounds described herein act as anti-microbial agents (e.g., anti-bacterials, anti-fungals, and anti-virals) by binding to the cellular membrane of bacteria and other microbes and inserting into the cell membrane, forming a pore that allows the leakage of ions and cytoplasmic materials that are critical to the microbe's survival, thereby leading to the death of the affected microbe. In addition, the CSA compounds described herein may also act to sensitize microbes to other types of antimicrobials. For example, at concentrations of the CSA compound below the corresponding minimum bacteriostatic concentration, CSAs have been shown to cause bacteria or fungi to become more susceptible to other antibiotics or antifungal agents, respectively, by increasing membrane permeability of the bacteria or fungi.

The charged groups are responsible for disrupting the bacterial or fungal cellular membrane, and without the charged groups, the CSA compound cannot disrupt the membrane to cause cell death or sensitization. Example of CSA compounds have a chemical structure of Formula I as shown below. As will be discussed in greater detail below, the R groups of Formula I can have a variety of different functionalities, thus providing a given ceragenin compound with specific, different properties. In addition, as will be appreciated by those of skill in the art, the sterol backbone can be formed of 5-member and/or 6-member rings, so that p, q, m, and n may independently be 1 (providing a 6-member ring) or 0 (providing a 5-member ring).

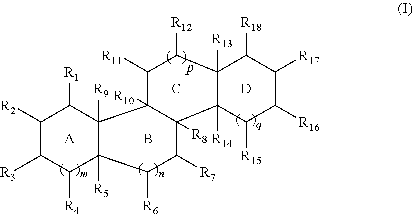

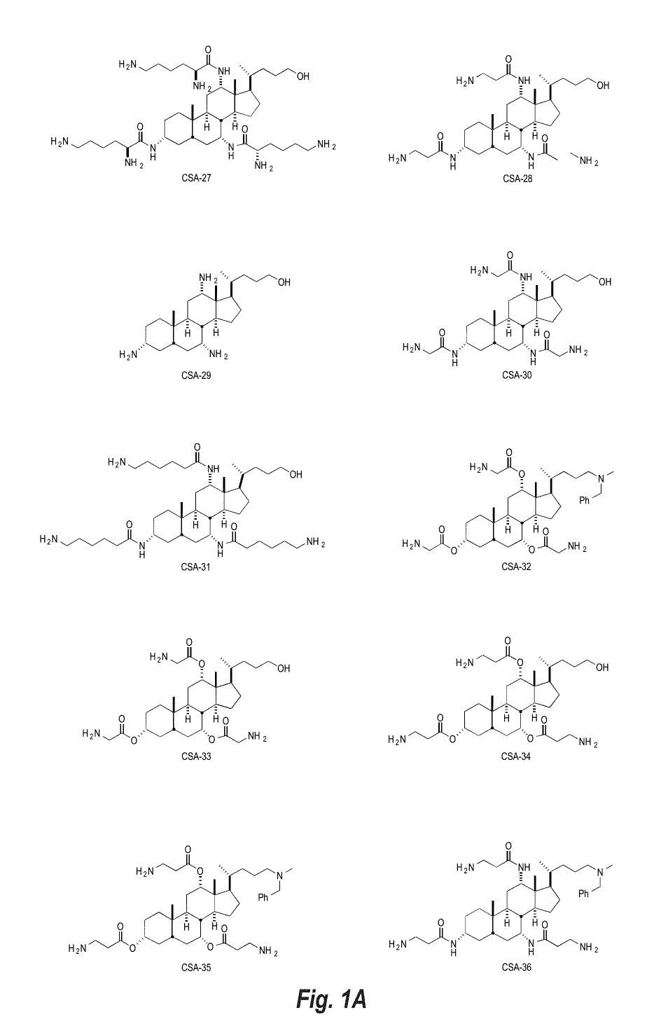

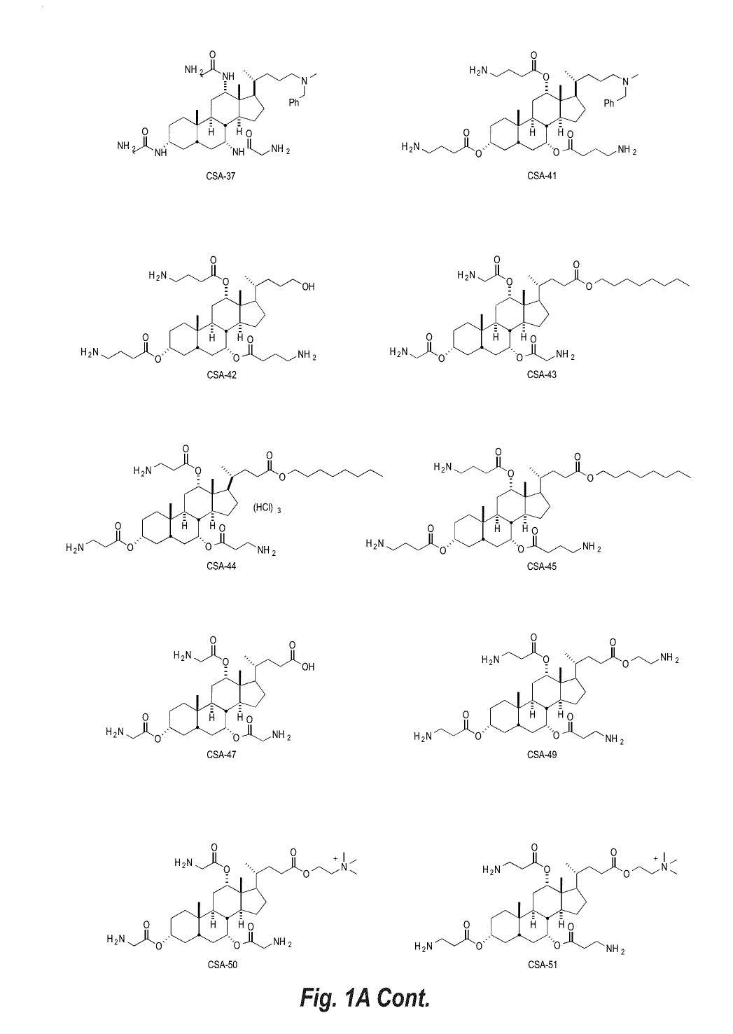

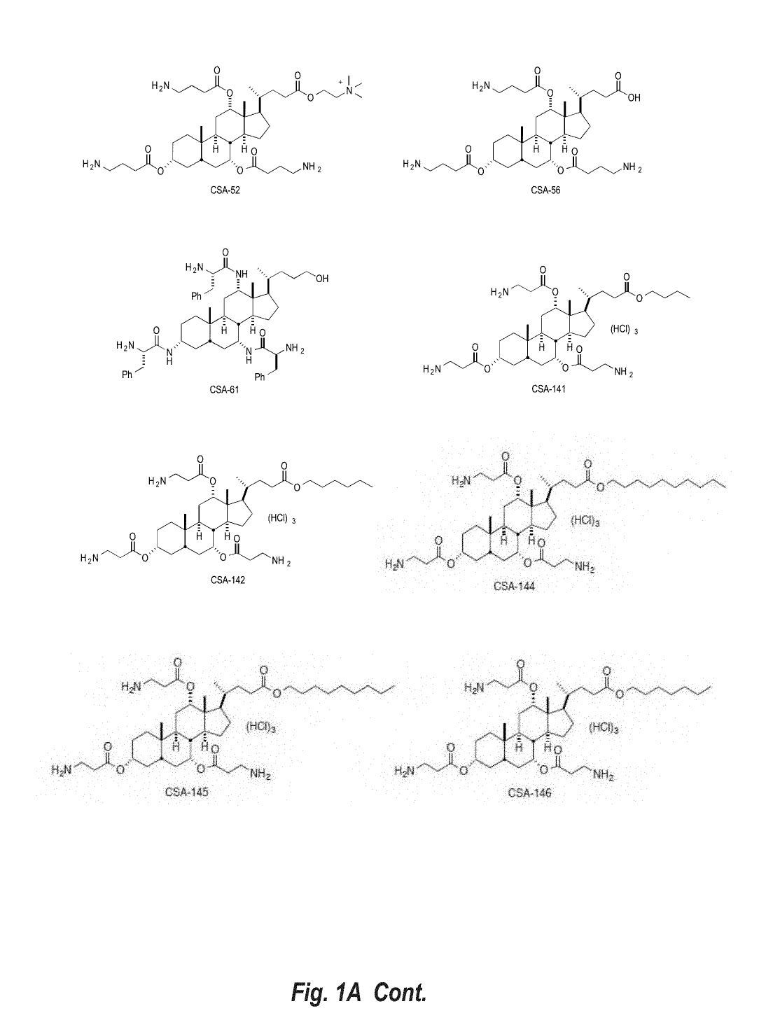

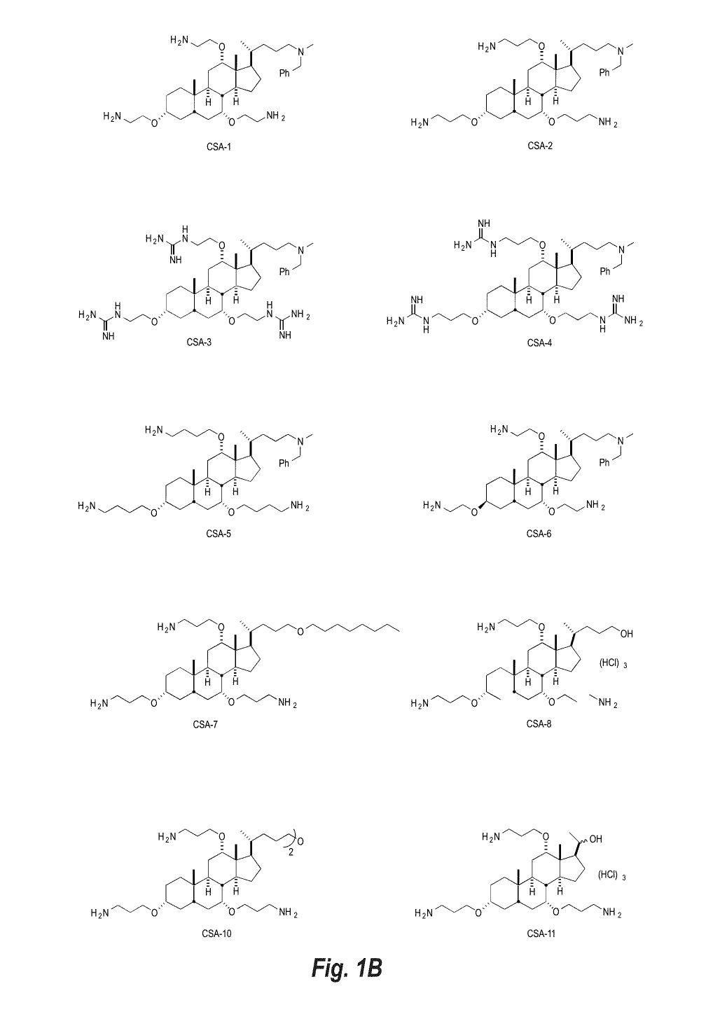

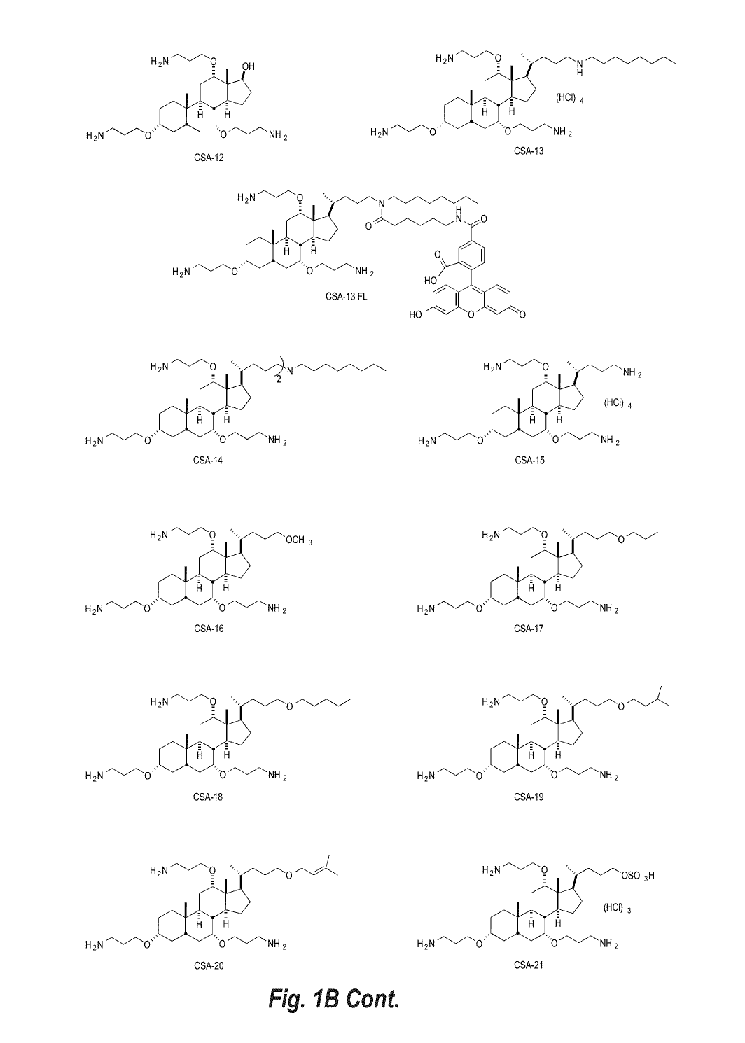

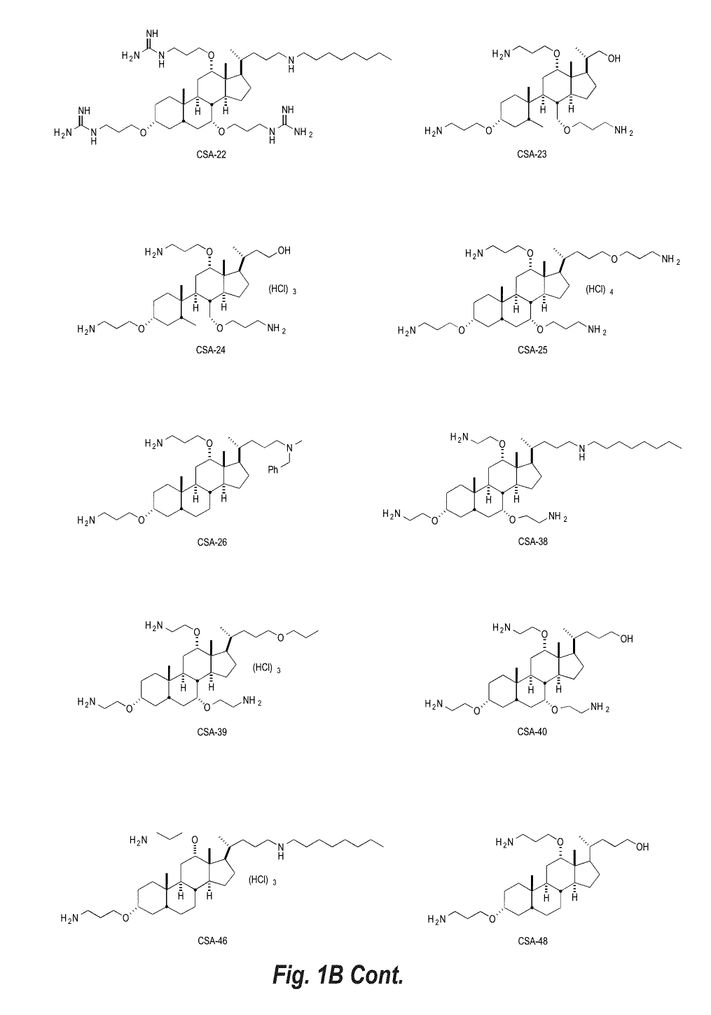

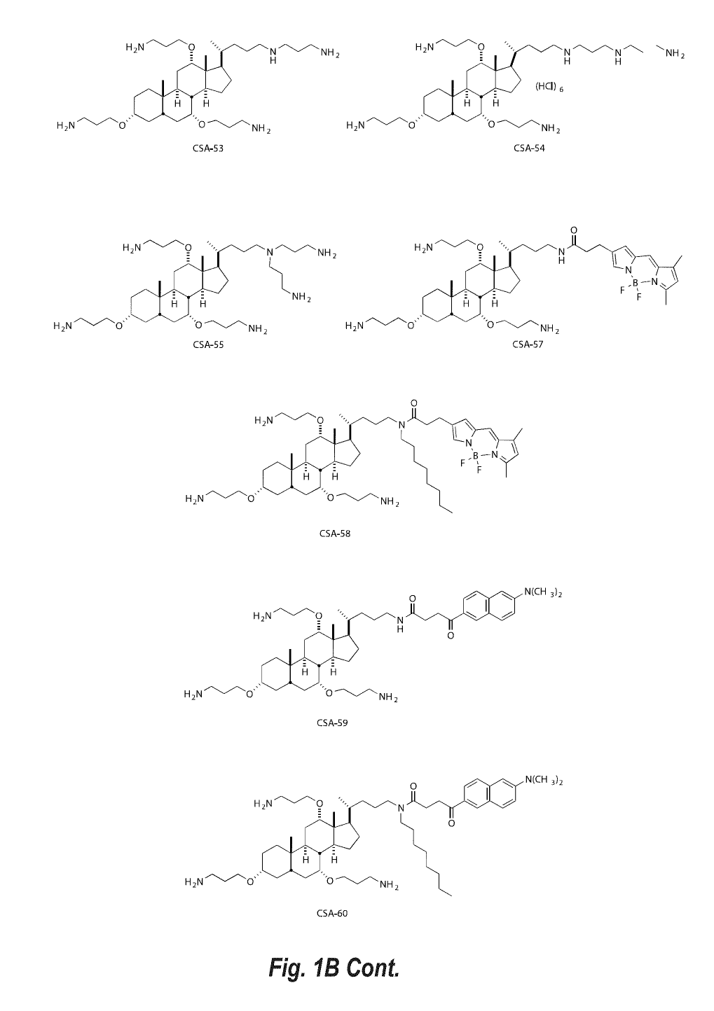

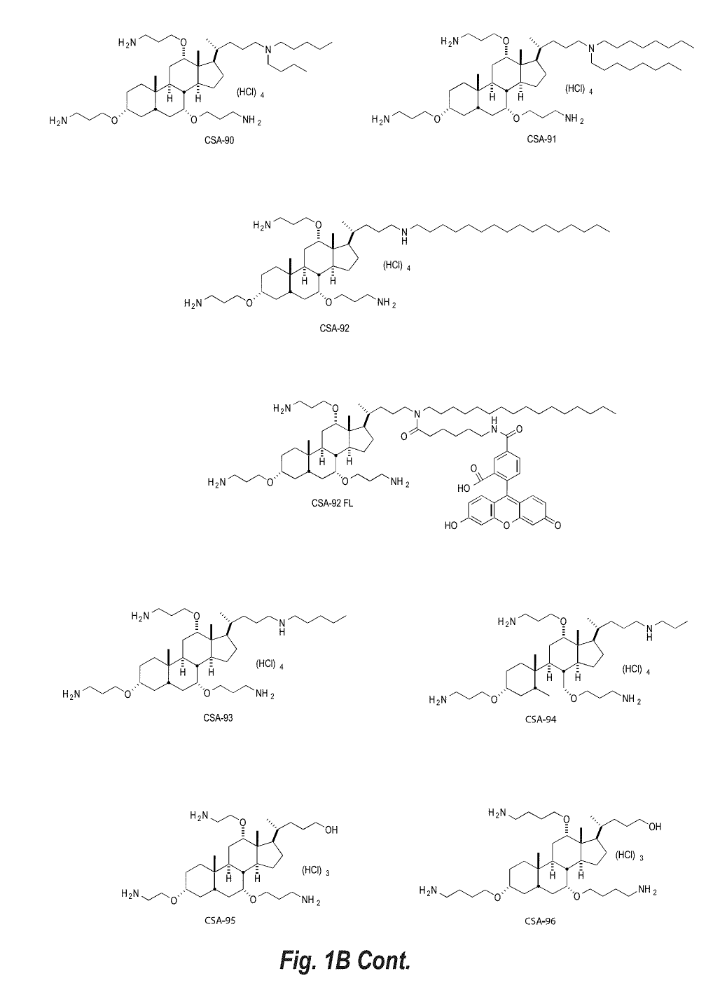

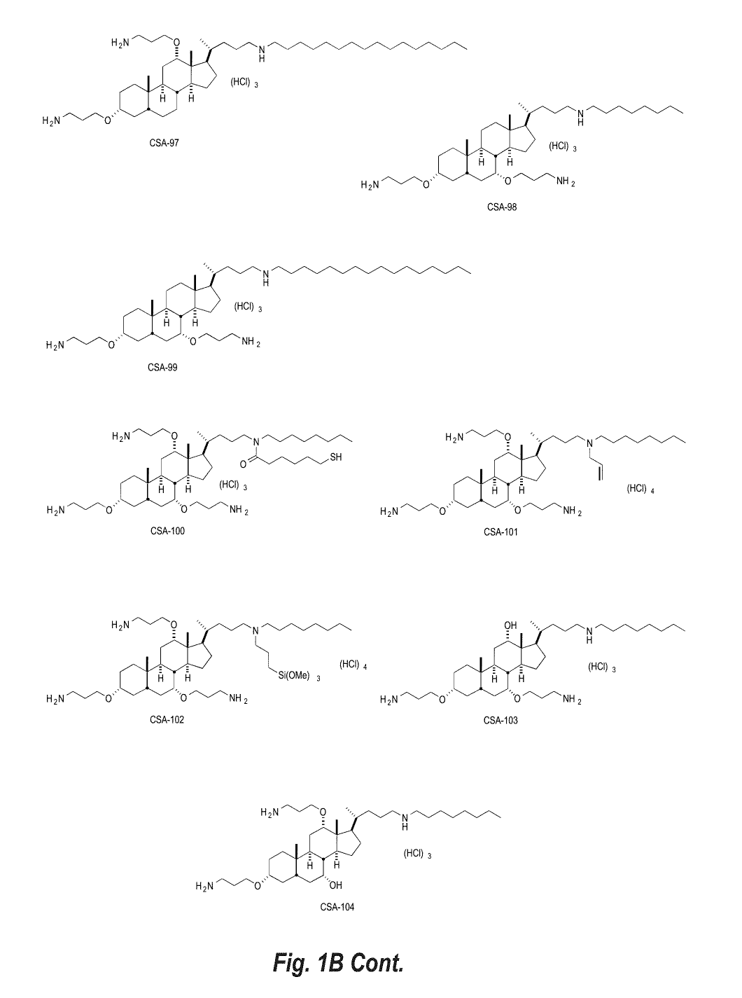

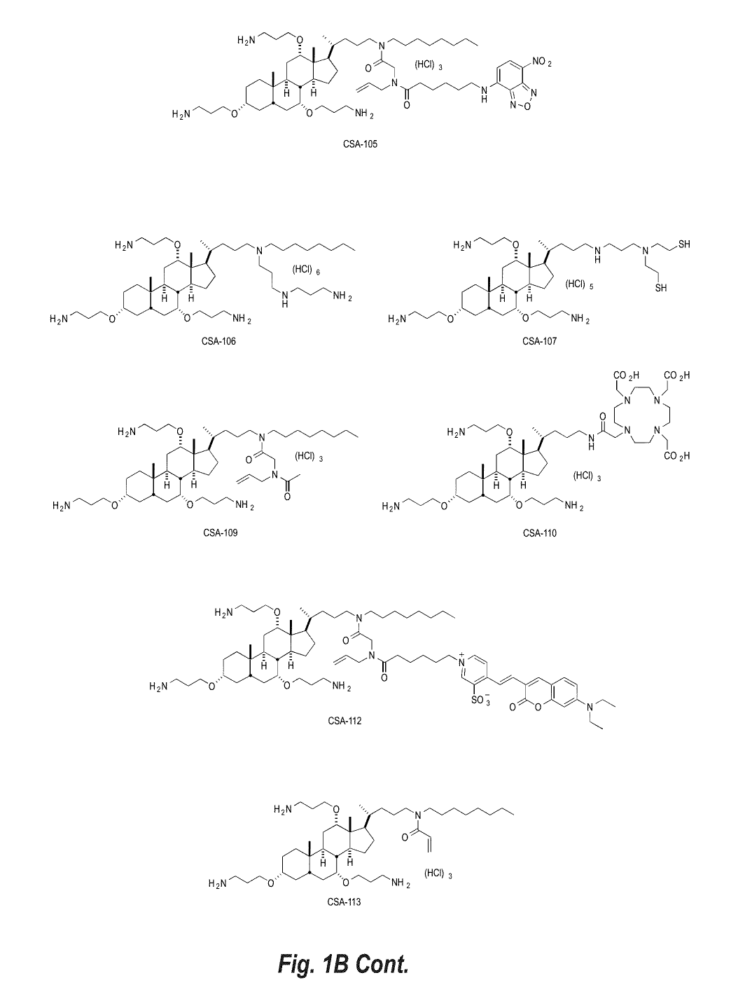

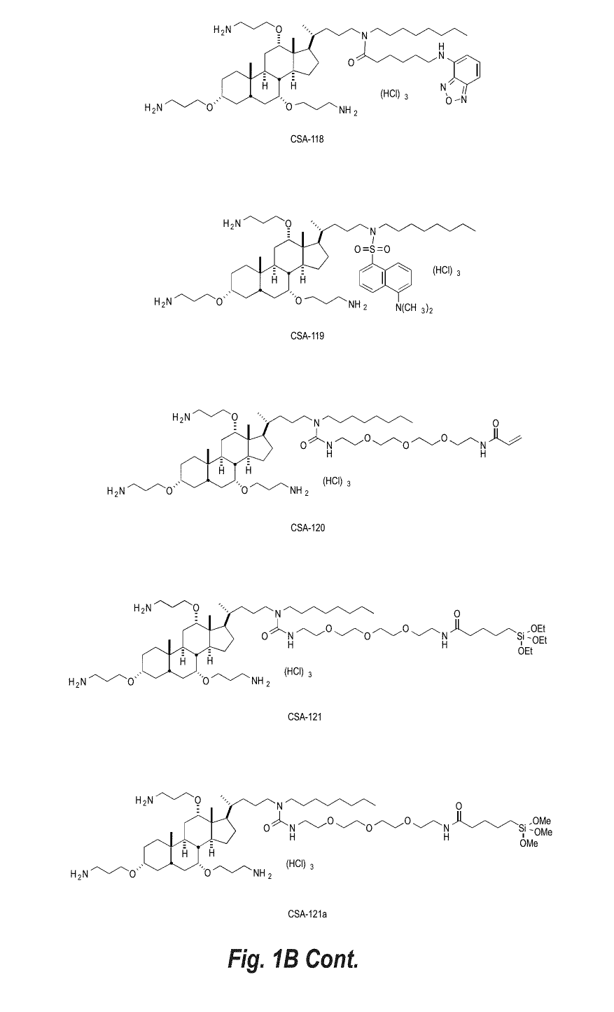

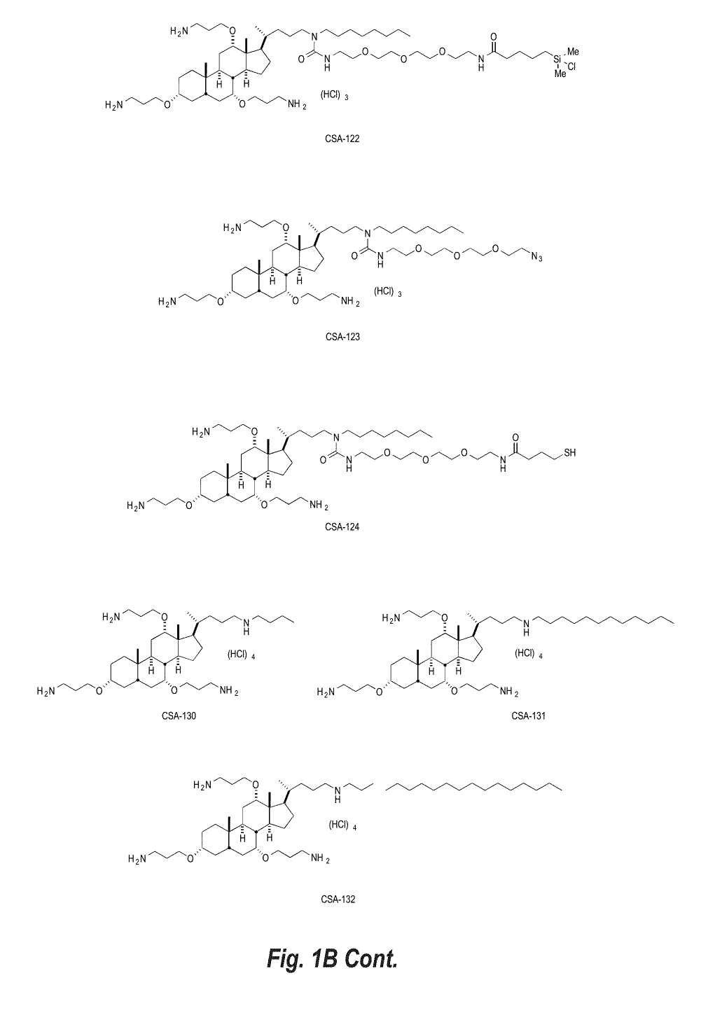

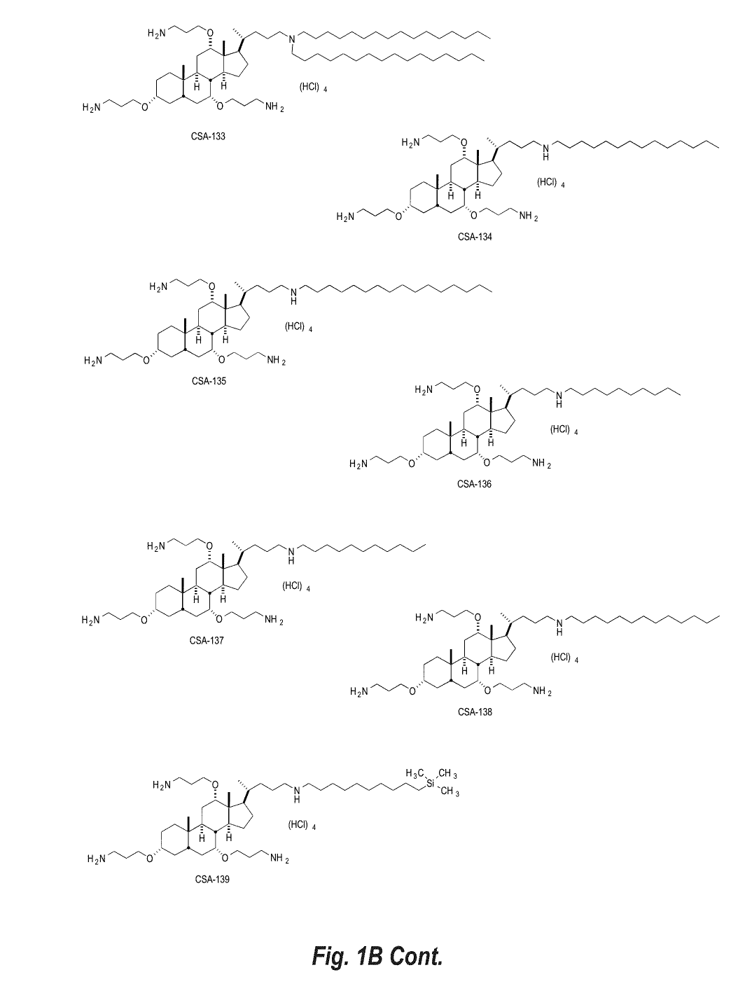

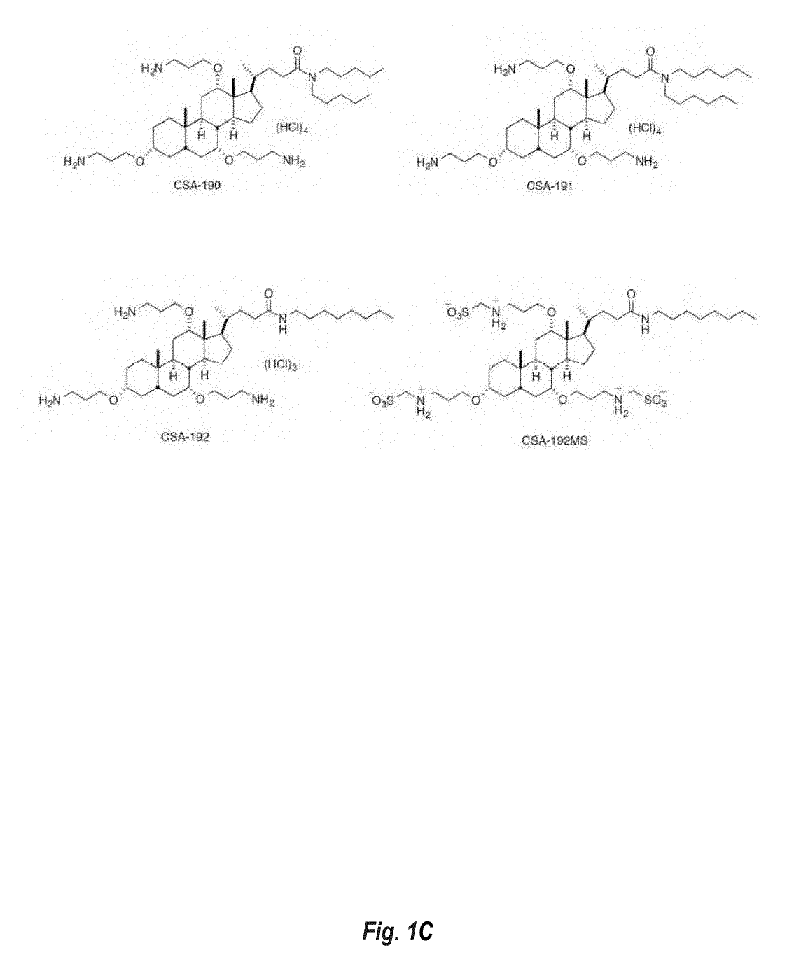

A number of examples of CSA compounds of Formula I that can be incorporated into the medical implants described herein are illustrated in FIGS. 1A-1C.

Typically, the CSAs of Formula I are of two types: (1) CSA compounds having cationic groups linked to the sterol backbone with hydrolysable linkages and (2) CSA compounds having cationic groups linked to the sterol backbone with non-hydrolysable linkages. For example, one type of hydrolysable linkage is an ester linkage, and one type of non-hydrolysable linkage is an ether linkage. CSA compounds of the first type can be “inactivated” by hydrolysis of the linkages coupling the cationic groups to the sterol backbone, whereas CSA compounds of the second type are more resistant to degradation and inactivation.

In some applications, it may be desirable for a medical implant to maintain antimicrobial effects for as long as possible. For example, relatively long-term use of medical devices such as catheters, IUDs, endotracheal tubes, and voice prostheses provides ample opportunity for fouling or introduction of infection. In many instances, the lifespan of these medical devices is essentially limited to how long they can resist fouling before becoming hazardous to the subject. Accordingly, enhancing the capability to resist microbial colonization and fouling for months, weeks, or even days can decrease medical equipment and/or medical care costs in addition to decreasing infection risks.

In other applications, the spreading of eluted CSA molecules beyond the implant site of the medical device may be a concern. Medical implant embodiments can be formed using an appropriate mixture of CSAs having hydrolysable and non-hydrolysable linkages to provide desired duration of CSA activity once the CSAs are exposed to biological conditions (e.g., once eluted from the medical implant).

A number of examples of compounds of Formula I that may be used in the embodiments described herein are illustrated in FIGS. 1A-1C. Examples of CSA compounds with non-hydrolysable linkages include, but are not limited to, CSA-1, CSA-26, CSA-38, CSA-40, CSA-46, CSA-48, CSA-53, CSA-55, CSA-57, CSA-60, CSA-90, CSA-107, CSA-109, CSA-110, CSA-112, CSA-113, CSA-118, CSA-124, CSA-130, CSA-131, CSA-139, CSA-190, CSA-191 and CSA-192. Suitable examples of CSA compounds with hydrolysable linkages include, but are not limited to CSA-27, CSA-28, CSA-29, CSA-30, CSA-31, CSA-32, CSA-33, CSA-34, CSA-35, CSA-36, CSA-37, CSA-41, CSA-42, CSA-43, CSA-44, CSA-45, CSA-47, CSA-49, CSA-50, CSA-51, CSA-52, CSA-56, CSA-61, CSA-141, CSA-142, CSA-144, CSA-145 and CSA-146. In a preferred embodiment, at least a portion of the CSA molecules incorporated into the medical device are CSA-131 or salt thereof (e.g., NDSA salt).

In some embodiments, the one or more CSA compounds may have a structure as shown in Formula I. In Formula I, at least two of R₃, R₇, or R₁₂ may independently include a cationic moiety attached to the Formula I structure via a hydrolysable (e.g., an ester) or non-hydrolizable (e.g., an ether) linkage. Optionally, a tail moiety may be attached to Formula I at R₁₈. The tail moiety may be charged, uncharged, polar, non-polar, hydrophobic, or amphipathic, for example, and can thereby be selected to adjust the properties of the CSA and/or to provide desired characteristics.

The anti-microbial activity of the CSA compounds can be affected by the orientation of the substituent groups attached to the backbone structure. In one embodiment, the substituent groups attached to the backbone structure are oriented on a single face of the CSA compound. Accordingly, each of R₃, R₇, and R₁₂ may be positioned on a single face of Formula I. In addition, R₁₈ may also be positioned on the same single face of Formula I.

In some embodiments, the CSA molecules are included by weight in a coating or a polymeric mixture at about 0.1%, 0.5%, 1%, 3%, 5%, 10%, 15%, 20%, 25%, or 30% or are included by weight within a range defined by any two of the foregoing values.

Another advantageous characteristic associated with one or more of the CSA compositions described herein is their effectiveness in preventing biofilms, including bacterial and/or fungal biofilms. Many other antimicrobial agents suitable for application to a live subject, including nearly all antibiotics, have limited effectiveness in killing fungi or bacteria present in a biofilm form. Microbes within biofilms are believed to be in something of a sessile state and are additionally protected by a relatively thick extracellular matrix. This results in the biofilm microbes surviving antimicrobial treatment, leaving them capable of continuing to pose a pathogenic threat even after treatment with such antimicrobials. CSA compounds, in contrast, have shown to be effective in killing biofilm microbes and in preventing the establishment and formation of biofilms.

In preferred embodiments, the CSA compounds used herein are provided in salt form. It has been found that certain salt forms of CSAs exhibit beneficial properties such as improved solubility, crystallinity, flow, and storage stability. Some embodiments are directed to a sulfuric acid addition salt or sulfonic acid addition salt of a CSA. In some embodiments, the sulfonic acid addition salt is a disulfonic acid addition salt. In some embodiments, the sulfonic acid addition salt is a 1,5-naphthalenedisulfonic acid (NDSA) addition salt, such as an NDSA salt of CSA-131 and/or an NDSA salt of CSA-192. In some embodiments, the acid addition salt is a mono-addition salt. In other embodiments, the acid addition salt is a di-addition salt. In other embodiments, the acid addition salt is a tetra-addition salt.

### II. Medical Implants Incorporating CSA Compounds

As used herein, an “implantable implant” refers to a medical device that may be implanted into a subject's tissues, deployed at a puncture or wound site, positioned for introducing or withdrawing material from a body cavity, or otherwise associated with a subject in such a way that biological compatibility is of concern (e.g., because infection and/or inflammation can result). It will be understood that such an implant need not be fully implanted within a subject's body. For example, portions of the implant may extend to areas external to the patient.

Non-limiting examples of medical implants which may incorporate one or more CSA compounds, as described herein, include catheters, vascular catheters, peritoneal dialysis catheters, urinary catheters, joint prostheses, penile implants, dialysis access devices, dialysis access grafts, hemodialysis devices, fistula devices, hemodialysis grafts, cardiac devices, prosthetic valves, pacemakers (including implantable cardioverter defibrillators, or ICDs, and vascular assist devices, or VADs), central nervous system devices (VPSs), endotracheal tubes, intravenous (IV) needles, IV feed lines, other IV components, feeder tubes, drains, prosthesis components (e.g., voice prostheses), peristaltic pumps, tympanostomy tubes, tracheostomy tubes, oral care devices (dentures, dental implants), intrauterine devices (IUDs), cardiac implants, and dermal fillers. The medical implants described herein may be provided for use with a human or animal patient/subject. They may be for short-term or long-term implantation. At least some of the embodiments described herein are particularly advantageous in applications where device biofouling, device rejection, and associated infection pathologies are common issues.

In some embodiments, a medical implant incorporates one or more CSA compounds by including a coating containing the CSA molecules. For example, an implantable medical device may be coated with a hydrogel material or other suitable coating carrier including the CSA molecules. In some embodiments, the hydrogel coating or other suitable coating provides a lubricious coating to the medical implant in addition to providing the beneficial anti-biofilm functionality of the CSA molecules.

In some embodiments, a medical implant additionally or alternatively incorporates one or more CSA compounds by including the CSA molecules within the structure of the medical implant itself. For example, the CSA molecules may be mixed with a moldable polymeric material prior to extruding, molding, or otherwise manufacturing the material to form at least a portion of the medical implant. In this manner, the implant includes a reservoir of CSA molecules directly incorporated into the structure of the implant to kill contacting microbes and/or prevent adherence and colonization of biofilm capable microbes.

The polymeric material of the medical implant may be any polymeric material with suitable biological compatibility for the intended use of the finished medical implant. In some embodiments, for example, the medical implant is formed at least partially from a silicone that has been mixed with the CSA molecules such that the CSA molecules are distributed within the silicone material.

The use of CSA compounds to prevent colonization of microbes and fouling of medical implants is unique in that CSAs can prevent both fungal and bacterial contamination. In general, the combination of fungal and bacteria growing on the same device leads to increased virulence and poor clinical outcomes, including higher rates of mortality. The CSA compounds unexpectedly provide a solution to this previously untreatable, or hard to treat, condition.

Any of the CSA compounds described herein may be utilized for incorporation with an implantable medical device. In some embodiments, CSA molecules are included in a salt form. Preferred salt forms include sulfuric acid addition salts or sulfonic acid addition salts, including NDSA addition salts such as 1,5-NDSA addition salts. These and other salt forms of CSAs have shown beneficial properties such as good flowability/mixability and storage stability. Further, these salt forms have been shown to have limited or no interaction with polymeric materials when mixed with the polymeric materials, leaving the CSA molecules in an active form capable of providing enhanced anti-biofilm functionality.

In some embodiments, the medical implant is formed at least partly of silicone. Silicone has shown good mixability with at least some of the CSA compounds disclosed herein, with no indication of the silicone reacting with or reducing the activity of the CSA molecules. Other polymers useful for making medical implants include polyethylene, polypropylene, polystyrene, polyester, polycarbonate, polyvinyl chloride, polyacrylate, polysulfone, polyvinylidene fluoride, polydimethylsiloxane, parylene, polyether ether ketone, polyamide, polytetrafluoroethylene, poly(methyl methacrylate), polyimide, polyurethane, other suitable biocompatible materials, and combinations thereof.

Medical implant embodiments described herein can provide a variety of benefits. For example, medical implants can have extended lifetimes as a result of preventing biofilm formation and associated fouling. Some implants, such as tracheostomy tubes, are typically required for months at a time, but must be replaced as fouling occurs. Extending the usable life of such medical devices reduces costs and reduces patient trauma and medical risks associated with removing and replacing the implant. Another example is a voice prosthesis. Such implants are intended to be permanent, yet they typically only last months at a time due to fungal and/or bacterial biofilm formation or colonization.

### III. Methods of Manufacturing a Medical Implant Incorporating CSA Compounds

In some embodiments, a method of manufacturing a medical implant with one or more incorporated CSA compounds comprises: (1) providing a biologically compatible moldable polymeric material; (2) mixing CSA molecules with the moldable polymeric material; and (3) forming the moldable polymeric material into medical implant.

In some embodiments, the CSA compounds are provided in a solid salt form. In some embodiments, solid form CSA compounds are processed to a desired average particle size prior to mixing with the moldable polymeric material, such as through a micronizing process using one or more impact mills (e.g., hammer mills, jet mills, and/or ball, pebble, or rod mills) or other suitable processing units. After sizing, the solid form CSA compounds will preferably have an average particle size of about 50 nm, 100 nm, 150 nm, 250 nm, 500 nm, 1 μm, or an average particle size within a range defined by any two of the foregoing values.

Medical implants manufactured so as to incorporate one or more CSA compounds within the structure of the implant are particularly beneficial in applications in which the medical device is intended to be in use for long periods of time, and/or where microbial colonization and fouling is a likely problem. For example, where embodiments utilizing a coating of CSA molecules may have about 5-10 days of efficacy, certain embodiments incorporating CSA molecules within the structure of the implant have shown efficacy over a time period of several months (e.g., 2-12 months, 3-9 months, or 4-6 months).

One or more embodiments are directed to methods of manufacturing a medical implant, the method comprising: (1) providing a medical implant; and (2) applying a coating to at least a portion of a surface of the medical implant to associate the coating with the medical implant, the coating being formulated with one or more CSA compounds.

In some embodiments, the coating can be a hydrogel formulated to provide the coating with lubricious properties. Hydrogels may be formed using one or more polymers such as polyvinyl alcohol, polyacrylic acid, polyethylene glycol, polyvinylpyrrolidone, polysaccharides, and polyacrylamide, for example. Hydrogels may be amorphous, semi-crystalline, or crystalline. In some embodiments, the hydrogel coating reduces the coefficient of friction at the surface of the medical device to which it is applied by up to about 5 times, 10 times, 15 times, 20 times, or 30 times.

### IV. Methods of Using a Medical Implant Incorporating CSA Compounds

One or more embodiments are directed to methods of preventing biofilm fouling, including biofilm fouling from bacterial and/or fungal biofilms, on a medical implant. In some embodiments, a method comprises: (1) providing a medical implant having one or more incorporated CSA compounds; (2) implanting the medical implant; and (3) the medical implant incorporating the CSA compounds killing microbes contacting the medical implant or preventing adherence of microbes contacting the medical implant to thereby prevent microbial colonization and fouling of the medical implant. The medical implant may be effective in killing and/or preventing adherence of a wide variety of microbes that would otherwise colonize, foul and/or form biofilms on or in the medical implant.

In some embodiments, the method provides enhanced protection from biofouling and/or associated infection (e.g., as compared to use of a similar medical implant not incorporating CSA compounds). The method can therefore provide increased efficacious lifespan of the medical.

In some embodiments, the CSA compounds in the medical implant maintain efficacy for preventing biofilm formation and fouling for at least 4 days after implantation, at least 7 days after implantation, at least 14 days after implantation, at least 30 days after implantation, at least 60 days after implantation, or about 90 days after implantation. In some embodiments, the medical implant maintains efficacy for as long as the implant resides at the implantation site (e.g., about a weeks, about two weeks, about a month, or about 2-3 months).

### V. EXAMPLES

### Example 1

A silicone-based Foley catheter was coated with a hydrogel coating of approximately 10 μm in thickness. The coating included CSA-131. The coating was initially shown to maintain efficacy for about 6-7 days.

### Example 2

A silicone-based Foley catheter was formed using silicone mixed with an NDSA salt form of CSA-131. The silicone catheter was shown to maintain high efficacy for the first three weeks, with test data showing efficacy lasting for at least 3-4 months.

### Example 3

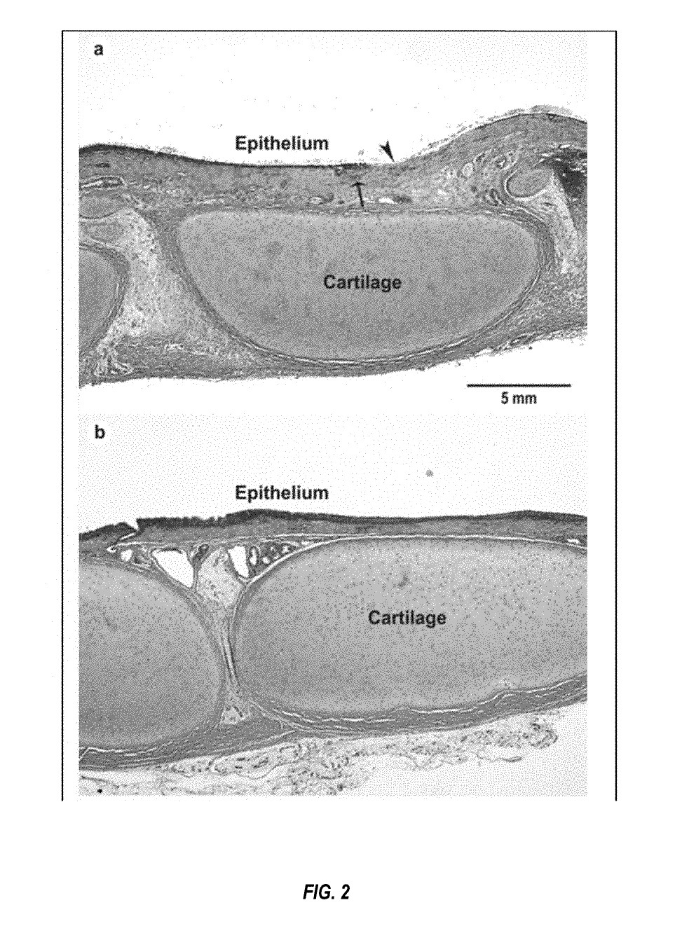

Pre-term lambs were intubated using endotracheal tubes (ETTs) including a coating having CSA-131. Tracheal mucosal integrity of the lambs was compared to the tracheal mucosal integrity of a control group (intubated with uncoated ETTs). The pre-term lambs intubated with coated ETTs showed markedly improved mucosal integrity compared to the pre-term lambs of the control group. FIG. 2 illustrates the histological appearance of tracheas of the premature lambs that were intubated for three days. Image (a) shows a trachea from a lamb intubated with an uncoated ETT. An area of denuded epithelium (arrowhead), and accumulation of white blood cells (arrow) are highlighted. Image (b) shows a trachea from a lamb intubated with an ETT coated with a CSA-131 containing coating. As shown, the epithelium is healthy and intact, and the subjacent connective tissue region is not inflamed.

### Example 4

Several CSA compounds were tested against Pseudomonas aeruginosa and Staphylococcus aureus mixed-species biofilms grown for an initial 22 hours and subjected to 20 hours of treatment. Many CSA compounds showed more potent anti-biofilm activity than the classical antimicrobial peptide (AMP) LL-37. Table 1 shows minimum inhibitory concentrations (MIC) and minimum bactericidal concentrations (MBC) of LL-37 and the various CSA compounds against the mixed-species biofilms.

TABLE 1

| | |
|---|---|
| P. aeruginosa (μg/ml) | S. aureus (μg/ml) |

| | MIC | MBC | MIC | MBC |
|---|---|---|---|---|
| LL-37 | >200 | >200 | >200 | >200 |
| CSA-8 | 25 | 100 | 3.125 | 6.25 |
| CSA-11 | >200 | >200 | 50 | 50 |
| CSA-13 | 3.125 | 6.25 | 0.78 | 0.78 |
| CSA-25 | 25 | 50 | 1.56 | 3.25 |
| CSA-44 | 3.125 | 6.25 | 1.56 | 3.25 |
| CSA-54 | 50 | 100 | 6.25 | 25 |
| CSA-90 | 6.25 | 6.25 | 1.56 | 1.56 |
| CSA-192 | 3.125 | 6.25 | 0.78 | 1.56 |
| CSA-131 | 3.125 | 3.125 | 0.78 | 1.56 |
| CSA-134 | 12.5 | 25 | 0.78 | 3.125 |
| CSA-138 | 3.125 | 6.25 | 1.56 | 3.125 |
| CSA-142 | 3.125 | 3.125 | 3.125 | 3.125 |
| CSA-144 | 12.5 | 50 | 3.125 | 3.125 |
| CSA-145 | 12.5 | 50 | 3.125 | 3.125 |

### Example 5

Segments of polyvinyl chloride (PVC) endotracheal tubes measuring 3 cm in length were placed in wells of a 6 well plate. 7 ml of inoculum were placed in each well. The process was repeated until all plates were prepared for the organisms listed in Table 2. Testing was repeated in triplicate for each test article. The samples were placed on a rotary shaker (110 revolutions per minute) and incubated at 37±2° C. for 24 hours. At the end of 24 hours the bacteria in the inoculum were characterized via serial dilution, plating, and counting. The endotracheal tube segment was also removed and neutralized. After neutralization, the adherent biofilm biomass was removed and characterized via serial dilution, plating, and counting.

As the number of viable organisms recovered from the treated catheters is expected to be low, a membrane filtration method provides an appropriate analysis for counting recovered organisms. The remaining recovery solution was filtered through a 0.45 μm filter membrane. Filters were placed on TSA/SDA/NA/TSBA plates and incubated at 37±2° C. and counted after sufficient incubation.

Log₁₀ reduction results are shown in Table 2 below. The log reduction is calculated by determining the Log₁₀ difference between uncoated devices and associated media and coated devices (using a polymer coating including CSA-131 at about 39.4 μg/cm²) and associated media. The log reduction is calculated as follows:

Log reduction(Media)=Control Log₁₀(CFU/ml)−Test Log₁₀(CFU/ml)

Log₁₀(CFU/ml)=Log₁₀(CFU/ml+1)

Log reduction(Device)=Control Log₁₀(CFU/device)−Test Log₁₀(CFU/device)

Log₁₀(CFU/device)=Log₁₀(CFU/device+1)

TABLE 2

| | | Log₁₀ Reduction | | |
|---|---|---|---|---|
| Organism | Source | Media | Device | Total |
| Pseudomonas | ATCC 27853 | 9.15 | 7.58 | 8.62 |
| aeruginosa | ATCC 10752 | 9.86 | 7.32 | 9.58 |
| | (clinical) | | | |
| | PA14 (clinical) | 7.24 | 5.81 | 7.00 |
| Staphylococcus | 456 (clinical) | 9.63 | 6.77 | 9.63 |
| aureus MRSA | 399 (clinical) | 8.89 | 6.24 | 8.23 |
| | U of C #18 | 9.36 | 5.92 | 8.44 |
| | (clinical) | | | |
| Klebsiella | 4352 (clinical) | 6.56 | 4.91 | 7.70 |
| pneumonia | ATCC 29011 | 6.76 | 3.80 | 6.82 |
| | 06-0140- | 9.49 | 5.21 | 7.85 |
| | M4505 | | | |

All results were statistically significant when compared to the uncoated control utilizing a non-pairwise, two-tailed Student's T-test (p≤0.05).

### Example 6

CSA-131 was tested in vitro against a set of clinical isolates representing bacterial species commonly associated with hospital-acquired bacterial pneumonia (HABP) or ventilator-associated bacterial pneumonia (VABP). Antimicrobial susceptibility testing for 74 clinical isolates was performed. Broth microdilution using frozen-form MIC panels consisted of three media types: cation-adjusted Mueller-Hinton broth (CA-HMB), CA-HMB supplemented with 2.5-5% lysed horse blood for S. pneumoniae and Haemophilus test media (HTM) for Haemophilus spp. Results are shown in Table 3.

TABLE 3

| | No. of isolates at MIC | | | | |
|---|---|---|---|---|---|
| Organisms | (cumulative % inhibited) | | | | |
| (No. tested) | 2 μg/ml | 4 μg/ml | 8 μg/ml | MIC₅₀ | MIC₉₀ |
| All (74) | 23 (31.1%) | 40 (85.1%) | 11 (100%) | 4 | 8 |
| Staphylococcus aureus | 10 (100%) | 0 (100%) | 0 (100%) | 2 | 2 |
| (10) | | | | | |
| Streptococcus Pneumoniae | 0 (0%) | 10 (100%) | 0 (100%) | 4 | 4 |
| (10) | | | | | |
| Haemophilus spp.^{a} (10) | 0 (0%) | 0 (0%) | 10 (100%) | 8 | 8 |
| Enterobacteriaceae^{b} (22) | 4 (18.2%) | 18 (100%) | 0 (100%) | 4 | 4 |
| Non-fermenters^{c} (22) | 9 (40.9%) | 12 (95.5%) | 1 (100%) | 4 | 4 |

^{a}includes 8 H. influenza and 2 H. parainfluenzae
^{b}includes 5 E. aerogenes, 5 E. cloacae species complex, 2 E. coli and 10 K. pneumoniae
^{c}includes 10 A. baumannii species complex, 10 P. aeruginosa and 2 S. maltophilia

### Example 7

Releases of CSA-131 from hydrogel-based coatings were investigated, including examining coating stability and reaction of the coating with ethylene oxide (ETO) during sterilization processes. Addition of CSA-131 HCl (10% relative to coating solids) resulted in some flaking of the coating from silicone tube segments after prolonged soaking in buffer. ETO treatment of tube segments coated with hydrogel containing CSA-131 HCl (10%) revealed substantial reaction, leading to ETO adducts of CSA-131 HCl. These reactions were reasoned to be due to residual nucleophilic activity of the amine groups in the CSA. To mask this reactivity, coatings were prepared including varies percentages of citric acid to form non-volatile salts of the CSA compound. ETO adduct formation was eliminated, and the mechanical integrity of the coating was improved, by the addition of at least 3% (by weight relative to hydrogel solids) citric acid.

### Example 8

A hydrogel coating containing 10% CSA-131 HCl and 3% citric acid, relative to the hydrogel solids, was tested for antibacterial efficacy using endotracheal tube segments (5.0 mm). These tube segments (surface area of ca. 1.5 cm²) were dip coated and cured to give coatings of approximately 10 microns. Coated tube segments were washed in phosphate buffered saline (PBS) for varied amounts of time before testing. Antibacterial testing involved immersing tube segments in 10% TSB in PBS (1.0 mL) and inoculating with Pseudomonas aeruginosa (PA01)@10⁶ colony forming units (CFU). Samples were incubated for 24 h, and bacterial growth in the medium was assayed visually (i.e., turbidity in the medium is caused by unrestricted bacterial growth). Previous studies have determined that if bacterial growth in surrounding media is inhibited then bacterial biofilms do not form on devices. And the converse is true: if growth is supported in the medium that the device is colonized, to some extent.

After 24 h of incubation, tube segments were immersed in fresh growth media (10% TSB in PBS, 1 mL), re-inoculated, and incubated. This procedure was repeated every 24 h until bacterial growth was observed in the growth medium for consecutive 24 h periods. Experiments were performed in triplicate. Results from these experiments are shown in Table 4. N=no growth, G=growth.

TABLE 4

| | Day | Day | Day | Day | Day | Day | Day |
|---|---|---|---|---|---|---|---|
| Wash Time | 1 | 2 | 3 | 4 | 5 | 6 | 7 |
| Unwashed | N | N | N | N | G | G | G |
| 15 min | N | N | N | N | G | G | G |
| 30 min | N | N | N | N | G | G | G |
| 1 hour | N | N | N | N | G | G | G |

### Example 9

An alternative salt form of CSA-131, a bis-naphthalene disulfonate salt (2-NDSA), was investigated, and found to form stable coatings with low CSA-131 2-NDSA solubility in water. Using a jet mill (Fluid Energy), CSA-131 2-NDSA was milled to an average particle size of 200 nm. These particles suspended well in 2-butanone (the solvent used in the coating application) and incorporated well into the hydrogel coatings. In this particulate form, CSA-131 2-NDSA did not interfere with polymer curing and did not react with ETO (that is, no ETO adducts were isolated after ETO sterilization).

Antibacterial activity of a hydrogel coating (ca. 10 microns) containing CSA-131 2-NDSA (10% by weight relative to hydrogel solids) was determined as described in Example 8 using methicillin-resistant Staphylococcus aureus (MRSA) and Pseudomonas aeruginosa (PA01). The coating prevented bacterial growth for eight days with MRSA and for seven days with PA01 as shown in Table 5.

TABLE 5

| Organism | Day 1 | Day 2 | Day 3 | Day 4 | Day 5 | Day 6 | Day 7 | Day 8 | Day 9 | Day 10 |
|---|---|---|---|---|---|---|---|---|---|---|
| MRSA | N | N | N | N | N | N | G | G | G | G |
| (1st rep.) | | | | | | | | | | |
| MRSA | N | N | N | N | N | N | N | N | G | G |
| (2nd rep.) | | | | | | | | | | |
| MRSA | N | N | N | N | N | N | N | N | G | G |
| (3rd rep.) | | | | | | | | | | |
| PA01 | N | N | N | N | N | N | N | G | G | G |
| (1st rep.) | | | | | | | | | | |
| PA01 | N | N | N | N | N | N | N | G | G | G |
| (2nd rep.) | | | | | | | | | | |
| PA01 | N | N | N | N | N | N | N | G | G | G |
| (3rd rep.) | | | | | | | | | | |

### Example 10

To better represent an implant environment where fluids flow through and/or around an implant (such as with endotracheal tubes), whole endotracheal tubes were coated with a hydrogel containing 10% CSA-131 2-NDSA and tested in a drip flow system. The tubes were placed at a 30° angle of declination and a nutrient medium (10% TSB in PBS) was flowed from the top to the bottom of the tube (1 mL/min) through the inner lumen of the tube. At eight-hour intervals, bacteria (PA01, 10⁶ CFU/mL) were passed through the tubes for 10 min (total of 10⁷ CFU) at each interval. At a time point 30 min after introduction of bacteria, the growth medium flowing through the tubes was collected and plated to determine the number of bacterial colonies present. After 24 h, tubes were removed and sections (5 mm) were cut from above and below the cuff. These were sonicated in a neutralizing medium and the resulting solution was plated to quantify bacterial biofilm growth. In both the collected effluent from the tubes and from sonication of tubes, no bacteria were detected (detection limit 10 CFU), demonstrating that the tubes remained sterile in the presence of three inoculations of 10⁷ bacteria in a growth medium.

### Example 11

The antifungal effectiveness of CSA-131 was tested against 100 Candida auris isolates. The C. auris isolates were from all over the world and covered known C. auris clades. The selection included isolated known to have elevated MIC values against fluconazole, the echinocandins, and/or amphotericin B.

Testing was performed according to the standards of the Clinical and Laboratory Standards Institute reference methodology M27-A3 (as of 2017). CSA-131 was dissolved in DMSO and diluted as described in M27-A3 to give a final DMSO concentration of <1%. Dilution plates were stored at −70° C. until used and were used within one week of being produced. All results were read visually after 24 hours of incubation at the lowest drug concentration at which there was a 50% decrease in growth by visual inspection. Quality control isolates C. parapsilosis ATCC 22019 and C. krusei ATCC 6258 were included on each day of testing although there are no standard QC values for these isolates against this compound. The MIC values for ATCC 22019 and ATCC 6258 remained within a tight range of 1-2 dilutions over the course of the study.

CSA-131 showed activity against all C. auris among this collection, with all isolates falling into one of two possible MIC values. The activity across the 4 clades was comparable. The MIC₅₀ value for this compound is not impacted by individual isolate status as echinocandin- or fluconazole-resistant. Results are shown in Tables 6 through 10.

TABLE 6
MIC data for 100 C. auris isolates

| | |
|---|---|
| Range | 0.5-1 |
| Mode | 1 |
| MIC₅₀ | 1 |
| MIC₉₀ | 1 |

TABLE 7
Distribution of MIC values

| MIC | No. of Isolates |
|---|---|
| <0.016 | 0 |
| 0.016 | 0 |
| 0.03 | 0 |
| 0.0625 | 0 |
| 0.125 | 0 |
| 0.25 | 0 |
| 0.5 | 39 |
| 1 | 61 |
| 2 | 0 |
| >2 | 0 |

TABLE 8
MIC values (μg/ml) for 100 C. auris isolates by clade

| | No. of isolates | Range | Mode | MIC₅₀ | MIC₉₀ |
|---|---|---|---|---|---|
| Clade 1 | 47 | 0.5-1 | 1 | 1 | 1 |
| Clade 2 | 11 | 0.5-1 | 1 | 1 | 1 |
| Clade 3 | 39 | 0.5-1 | 1 | 1 | 1 |
| Clade 4 | 3 | 0.5-1 | 0.5 | 0.5 | 1 |

TABLE 9
MIC data for fluconazole resistant vs. susceptible isolates

| | |
|---|---|
| Fluconazole | MIC (μg/ml) |

| posture | Susceptible | Resistant |
|---|---|---|
| No. of isolates | 30 | 69 |
| Range | 0.5-1 | 0.5-1 |
| Mode | N/A | 1 |
| MIC₅₀ | 0.5 | 1 |
| MIC₉₀ | 1 | 1 |

TABLE 10
MIC data for isolates with elevated echinocandin MICs

| | |
|---|---|
| No. of isolates | 7 |
| Range | 0.5-1 |
| Mode | 1 |
| MIC₅₀ | 1 |
| MIC₉₀ | 1 |

### VI. Additional Details of CSA Compounds

More specific examples of CSA compounds according to Formula I are shown below in Formulas II and III, wherein Formula III differs from Formula II by omitting R₁₅ and the ring carbon to which it is attached. The R groups shown in the Formulae can have a variety of different structures. CSA compounds, and a variety of different R groups, useful in accordance with the present disclosure, are disclosed in U.S. Pat. Nos. 6,350,738, 6,486,148, 6,767,904, 7,598,234, 7,754,705, 8,975,310 and 9,434,759, which are incorporated herein by reference.

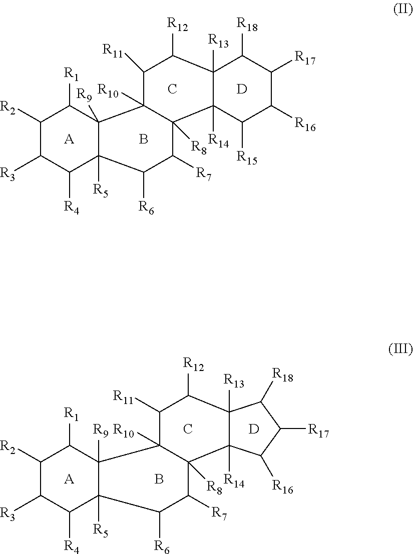

In some embodiments of Formulas II and III, at least two of R₃, R₇, and R₁₂ may independently include a cationic moiety (e.g., amino or guanidino groups) bonded to the steroid backbone structure via a non-hydrolysable or hydrolysable linkage. For the embodiments of the present disclosure, the linkage is preferably non-hydrolysable under conditions of sterilization and storage, and physiological conditions. Such cationic functional groups (e.g., amino or guanidino groups) may be separated from the backbone by at least one, two, three, four or more atoms.

Optionally, a tail moiety may be attached to the backbone structures at R₁₈. The tail moiety may have variable chain length or size and may be charged, uncharged, polar, non-polar, hydrophobic, amphipathic, and the like. The tail moiety may, for example, be configured to alter the hydrophobicity/hydrophilicity of the ceragenin compound. CSA compounds of the present disclosure having different degrees of hydrophobicity/hydrophilicity may, for example, have different rates of uptake into different target microbes.

The R groups described herein, unless specified otherwise, may be substituted or unsubstituted.

In some embodiments shown by Formulas II and III:

each of fused rings A, B, C, and D may be independently saturated, or may be fully or partially unsaturated, provided that at least two of A, B, C, and D are saturated, wherein rings A, B, C, and D form a ring system. Other ring systems can also be used, e.g., 5-member fused rings and/or compounds with backbones having a combination of 5- and 6-membered rings;

R₁ through R₄, R₆, R₇, R₁₁, R₁₂, R₁₅, R₁₆, and R₁₈ are independently selected from the group consisting of hydrogen, hydroxyl, alkyl, hydroxyalkyl, alkyloxyalkyl, alkylcarboxyalkyl, alkylaminoalkyl, alkylaminoalkylamino, alkylaminoalkylamino-alkylamino, aminoalkyl, aryl, arylaminoalkyl, haloalkyl, alkenyl, alkynyl, oxo, a linking group attached to a second steroid, aminoalkyloxy, aminoalkyloxyalkyl, aminoalkylcarboxy, aminoalkylaminocarbonyl, aminoalkylcarboxamido, di(alkyl)aminoalkyl, H₂N—HC(Q₅)—C(O)—O—, H₂N—HC(Q₅)—C(O)—N(H)—, azidoalkyloxy, cyanoalkyloxy, P.G.-HN—HC(Q₅)—C(O)—O—, guanidinoalkyloxy, quaternary ammonium alkylcarboxy, and guanidinoalkyl carboxy, where Q₅ is a side chain of any amino acid (including a side chain of glycine, i.e., H), and P.G. is an amino protecting group; and

R₅, R₈, R₉, R₁₀, R₁₃, R₁₄ and R₁₈ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, alkyl, hydroxyalkyl, alkyloxyalkyl, aminoalkyl, aryl, haloalkyl, alkenyl, alkynyl, oxo, a linking group attached to a second steroid, aminoalkyloxy, aminoalkylcarboxy, aminoalkylaminocarbonyl, di(alkyl)aminoalkyl, H₂N—HC(Q₅)—C(O)—O—, H₂N—HC(Q₅)—C(O)—N(H)—, azidoalkyloxy, cyanoalkyloxy, P.G.-HN—HC(Q₅)—C(O)—O—, guanidinoalkyloxy, and guanidinoalkyl-carboxy, where Q₅ is a side chain of any amino acid, P.G. is an amino protecting group.

In some embodiments, at least one, and sometimes two or three of R₁₋₄, R₆, R₇, R₁₁, R₁₂, R₁₅, R₁₆, R₁₇, and R₁₈ are independently selected from the group consisting of aminoalkyl, aminoalkyloxy, alkylcarboxyalkyl, alkyl aminoalkyl amino, alkyl aminoalkyl-aminoalkylamino, aminoalkylcarboxy, arylaminoalkyl, aminoalkyloxyaminoalkylamino-carbonyl, aminoalkylaminocarbonyl, aminoalkyl-carboxyamido, a quaternary ammonium alkylcarboxy, di(alkyl)aminoalkyl, H₂N—HC(Q₅)—C(O)—O—, H₂N—HC(Q₅)—C(O)—N(H)—, azidoalkyloxy, cyanoalkyloxy, P.G.-HN—HC(Q₅)—C(O)—O—, guanidine-alkyloxy, and guanidinoalkylcarboxy.

In some embodiments, R₁ through R₄, R₆, R₇, R₁₁, R₁₂, R₁₅, R₁₆, and R₁₈ are independently selected from the group consisting of hydrogen, hydroxyl, (C₁-C₂₂) alkyl, (C₁-C₂₂) hydroxyalkyl, (C₁-C₂₂) alkyloxy-(C₁-C₂₂) alkyl, (C₁-C₂₂) alkylcarboxy-(C₁-C₂₂) alkyl, (C₁-C₂₂) alkylamino-(C₁-C₂₂) alkyl, (C₁-C₂₂) alkylamino-(C₁-C₂₂) alkylamino, (C₁-C₂₂) alkylamino-(C₁-C₂₂) alkylamino-(C₁-C₂₂) alkylamino, (C₁-C₂₂) aminoalkyl, aryl, arylamino-(C₁-C₂₂) alkyl, (C₁-C₂₂) haloalkyl, C₂-C₆ alkenyl, C₂-C₆ alkynyl, oxo, a linking group attached to a second steroid, (C₁-C₂₂) aminoalkyloxy, (C₁-C₂₂) aminoalkyloxy-(C₁-C₂₂) alkyl, (C₁-C₂₂) aminoalkylcarboxy, (C₁-C₂₂) aminoalkylaminocarbonyl, (C₁-C₂₂) aminoalkyl-carboxamido, di(C₁-C₂₂ alkyl)aminoalkyl, H₂N—HC(Q₅)—C(O)—O—, H₂N—HC(Q₅)—C(O)—N(H)—, (C₁-C₂₂) azidoalkyloxy, (C₁-C₂₂) cyanoalkyloxy, P.G.-HN—HC(Q₅)—C(O)—O—, (C₁-C₂₂) guanidinoalkyloxy, (C₁-C₂₂) quaternary ammonium alkylcarboxy, and (C₁-C₂₂) guanidinoalkyl carboxy, where Q₅ is a side chain of an amino acid (including a side chain of glycine, i.e., H), and P.G. is an amino protecting group; and

R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₂₂) alkyl, (C₁-C₂₂) hydroxyalkyl, (C₁-C₂₂) alkyloxy-(C₁-C₂₂) alkyl, (C₁-C₂₂) aminoalkyl, aryl, (C₁-C₂₂) haloalkyl, (C₂-C₆) alkenyl, (C₂-C₆) alkynyl, oxo, a linking group attached to a second steroid, (C₁-C₂₂) aminoalkyloxy, (C₁-C₂₂) aminoalkylcarboxy, (C₁-C₂₂) aminoalkylaminocarbonyl, di(C₁-C₂₂ alkyl)aminoalkyl, H₂N—HC(Q₅)—C(O)—O—, H₂N—HC(Q₅)—C(O)—N(H)—, (C₁-C₂₂) azidoalkyloxy, (C₁-C₂₂) cyanoalkyloxy, P.G.-HN—HC(Q₅)—C(O)—O—, (C₁-C₂₂) guanidinoalkyloxy, and (C₁-C₂₂) guanidinoalkylcarboxy, where Q5 is a side chain of any amino acid, and P.G. is an amino protecting group;

provided that at least two or three of R₁₋₄, R₆, R₇, R₁₁, R₁₂, R₁₅, R₁₆, R₁₇, and R₁₈ are independently selected from the group consisting of (C₁-C₂₂) aminoalkyl, (C₁-C₂₂) aminoalkyloxy, (C₁-C₂₂) alkylcarboxy-(C₁-C₂₂) alkyl, (C₁-C₂₂) alkylamino-(C₁-C₂₂) alkylamino, (C₁-C₂₂) alkylamino-(C₁-C₂₂) alkylamino (C₁-C₂₂) alkylamino, (C₁-C₂₂) aminoalkylcarboxy, arylamino (C₁-C₂₂) alkyl, (C₁-C₂₂) aminoalkyloxy (C₁-C₂₂) aminoalkylaminocarbonyl, (C₁-C₂₂) aminoalkylaminocarbonyl, (C₁-C₂₂) aminoalkylcarboxyamido, (C₁-C₂₂) quaternary ammonium alkylcarboxy, di(C₁-C₂₂ alkyl)aminoalkyl, H₂N—HC(Q₅)—C(O)—O—, H₂N—HC(Q₅)—C(O)—N(H)—, (C₁-C₂₂) azidoalkyloxy, (C₁-C₂₂) cyanoalkyloxy, P.G.-HN—HC(Q₅)—C(O)—O—, (C₁-C₂₂) guanidinoalkyloxy, and (C₁-C₂₂) guanidinoalkylcarboxy.

In some embodiments, R₁ through R₄, R₆, R₇, R₁₁, R₁₂, R₁₅, R₁₆, and R₁₈ are independently selected from the group consisting of hydrogen, hydroxyl, (C₁-C₁₈) alkyl, (C₁-C₁₈) hydroxyalkyl, (C₁-C₁₈) alkyloxy-(C₁-C₁₈) alkyl, (C₁-C₁₈) alkylcarboxy-(C₁-C₁₈) alkyl, (C₁-C₁₈) alkylamino-(C₁-C₁₈)alkyl, (C₁-C₁₈) alkylamino-(C₁-C₁₈) alkylamino, (C₁-C₁₈) alkylamino-(C₁-C₁₈) alkylamino-(C₁-C₁₈) alkylamino, (C₁-C₁₈) aminoalkyl, aryl, arylamino-(C₁-C₁₈) alkyl, oxo, (C₁-C₁₈) aminoalkyloxy, (C₁-C₁₈) aminoalkyloxy-(C₁-C₁₈) alkyl, (C₁-C₁₈) aminoalkylcarboxy, (C₁-C₁₈) aminoalkylaminocarbonyl, (C₁-C₁₈) aminoalkyl-carboxamido, di(C₁-C₁₈ alkyl)aminoalkyl, (C₁-C₁₈) guanidinoalkyloxy, (C₁-C₁₈) quaternary ammonium alkylcarboxy, and (C₁-C₁₈) guanidinoalkyl carboxy; and

R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₁₈) alkyl, (C₁-C₁₈) hydroxyalkyl, (C₁-C₁₈) alkyloxy-(C₁-C₁₈) alkyl, (C₁-C₁₈) alkylcarboxy-(C₁-C₁₈) alkyl, (C₁-C₁₈) alkylamino-(C₁-C₁₈)alkyl, (C₁-C₁₈) alkylamino-(C₁-C₁₈) alkylamino, (C₁-C₁₈) alkylamino-(C₁-C₁₈) alkylamino-(C₁-C₁₈) alkylamino, (C₁-C₁₈) aminoalkyl, aryl, arylamino-(C₁-C₁₈) alkyl, oxo, (C₁-C₁₈) aminoalkyloxy, (C₁-C₁₈) aminoalkyloxy-(C₁-C₁₈) alkyl, (C₁-C₁₈) aminoalkylcarboxy, (C₁-C₁₈) aminoalkylaminocarbonyl, (C₁-C₁₈) aminoalkylcarboxamido, di(C₁-C₁₈alkyl)aminoalkyl, (C₁-C₁₈) guanidinoalkyloxy, (C₁-C₁₈) quaternary ammonium alkylcarboxy, and (C₁-C₁₈) guanidinoalkyl carboxy,

provided that at least two or three of R₁₋₄, R₆, R₇, R₁₁, R₁₂, R₁₅, R₁₆, R₁₇, and R₁₈ are independently selected from the group consisting of of hydrogen, hydroxyl, an unsubstituted (C₁-C₁₈) alkyl, unsubstituted (C₁-C₁₈) hydroxyalkyl, unsubstituted (C₁-C₁₈) alkyloxy-(C₁-C₁₈) alkyl, unsubstituted (C₁-C₁₈) alkylcarboxy-(C₁-C₁₈) alkyl, unsubstituted (C₁-C₁₈) alkylamino-(C₁-C₁₈)alkyl, unsubstituted (C₁-C₁₈) alkylamino-(C₁-C₁₈) alkylamino, unsubstituted (C₁-C₁₈) alkylamino-(C₁-C₁₈) alkylamino-(C₁-C₁₈) alkylamino, an unsubstituted (C₁-C₁₈) aminoalkyl, an unsubstituted aryl, an unsubstituted arylamino-(C₁-C₁₈) alkyl, oxo, an unsubstituted (C₁-C₁₈) aminoalkyloxy, an unsubstituted (C₁-C₁₈) aminoalkyloxy-(C₁-C₁₈) alkyl, an unsubstituted (C₁-C₁₈) aminoalkylcarboxy, an unsubstituted (C₁-C₁₈) aminoalkylaminocarbonyl, an unsubstituted (C₁-C₁₈) aminoalkylcarboxamido, an unsubstituted di(C₁-C₁₈alkyl)aminoalkyl, unsubstituted (C₁-C₁₈) guanidinoalkyloxy, unsubstituted (C₁-C₁₈) quaternary ammonium alkylcarboxy, and unsubstituted (C₁-C₁₈) guanidinoalkyl carboxy.

In some embodiments, R₃, R₇, R₁₂, and R₁₈ are independently selected from the group consisting of hydrogen, an unsubstituted (C₁-C₁₈) alkyl, unsubstituted (C₁-C₁₈) hydroxyalkyl, unsubstituted (C₁-C₁₈) alkyloxy-(C₁-C₁₈) alkyl, unsubstituted (C₁-C₁₈) alkylcarboxy-(C₁-C₁₈) alkyl, unsubstituted (C₁-C₁₈) alkylamino-(C₁-C₁₈)alkyl, unsubstituted (C₁-C₁₈) alkylamino-(C₁-C₁₈) alkylamino, unsubstituted (C₁-C₁₈) alkylamino-(C₁-C₁₈) alkylamino-(C₁-C₁₈) alkylamino, an unsubstituted (C₁-C₁₈) aminoalkyl, an unsubstituted arylamino-(C₁-C₁₈) alkyl, an unsubstituted (C₁-C₁₈) aminoalkyloxy, an unsubstituted (C₁-C₁₈) aminoalkyloxy-(C₁-C₁₈) alkyl, an unsubstituted (C₁-C₁₈) aminoalkylcarboxy, an unsubstituted (C₁-C₁₈) aminoalkylaminocarbonyl, an unsubstituted (C₁-C₁₈) aminoalkylcarboxamido, an unsubstituted di(C₁-C₁₈ alkyl)aminoalkyl, unsubstituted (C₁-C₁₈) guanidinoalkyloxy, unsubstituted (C₁-C₁₈) quaternary ammonium alkylcarboxy, and unsubstituted (C₁-C₁₈) guanidinoalkyl carboxy.

In some embodiments, R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆) alkyl.

In some embodiments, R₃, R₇, R₁₂, and R₁₈ are independently selected from the group consisting of hydrogen, an unsubstituted (C₁-C₆) alkyl, unsubstituted (C₁-C₁₆) hydroxyalkyl, unsubstituted (C₁-C₁₆) alkyloxy-(C₁-C₅) alkyl, unsubstituted (C₁-C₁₆) alkylcarboxy-(C₁-C₅) alkyl, unsubstituted (C₁-C₁₆) alkylamino-(C₁-C₅)alkyl, (C₁-C₁₆) alkylamino-(C₁-C₅) alkylamino, unsubstituted (C₁-C₁₆) alkylamino-(C₁-C₁₆) alkylamino-(C₁-C₅) alkylamino, an unsubstituted (C₁-C₁₆) aminoalkyl, an unsubstituted arylamino-(C₁-C₅) alkyl, an unsubstituted (C₁-C₅) aminoalkyloxy, an unsubstituted (C₁-C₁₆) aminoalkyloxy-(C₁-C₅) alkyl, an unsubstituted (C₁-C₅) aminoalkylcarboxy, an unsubstituted (C₁-C₅) aminoalkylaminocarbonyl, an unsubstituted (C₁-C₅) aminoalkylcarboxamido, an unsubstituted di(C₁-C₅ alkyl)amino-(C₁-C₅) alkyl, unsubstituted (C₁-C₅) guanidinoalkyloxy, unsubstituted (C₁-C₁₆) quaternary ammonium alkylcarboxy, and unsubstituted (C₁-C₁₆) guanidinoalkylcarboxy.

In some embodiments, R₁, R₂, R₄, R₅, R₆, R₈, R₁₀, R₁₁, R₁₄, R₁₆, and R₁₇ are each hydrogen; and R₉ and R₁₃ are each methyl.

In some embodiments, R₃, R₇, R₁₂, and R₁₈ are independently selected from the group consisting of aminoalkyloxy; aminoalkylcarboxy; alkylaminoalkyl; alkoxycarbonylalkyl; alkylcarbonylalkyl; di(alkyl)aminoalkyl; alkylcarboxyalkyl; and hydroxyalkyl.

In some embodiments, R₃, R₇, and R₁₂ are independently selected from the group consisting of aminoalkyloxy and aminoalkylcarboxy; and R₁₈ is selected from the group consisting of alkylaminoalkyl; alkoxycarbonylalkyl; alkylcarbonyloxyalkyl; di(alkyl)aminoalkyl; alkylaminoalkyl; alkyoxycarbonylalkyl; alkylcarboxyalkyl; and hydroxyalkyl.

In some embodiments, R₃, R₇, and R₁₂ are the same.

In some embodiments, R₃, R₇, and R₁₂ are aminoalkyloxy.

In some embodiments, R₁₈ is alkylaminoalkyl.

In some embodiments, R₁₈ is alkoxycarbonylalkyl.

In some embodiments, R₁₈ is di(alkyl)aminoalkyl.

In some embodiments, R₁₈ is alkylcarboxyalkyl.

In some embodiments, R₁₈ is hydroxyalkyl.

In some embodiments, R₃, R₇, and R₁₂ are aminoalkylcarboxy.

In some embodiments, R₃, R₇, R₁₂, and R₁₈ are independently selected from the group consisting of aminoalkyloxy; aminoalkylcarboxy; alkylaminoalkyl; di-(alkyl)aminoalkyl; alkoxycarbonylalkyl; and alkylcarboxyalkyl.

In some embodiments, R₃, R₇, and R₁₂ are independently selected from the group consisting of aminoalkyloxy and aminoalkylcarboxy, and wherein R₁₈ is selected from the group consisting of alkylaminoalkyl; di-(alkyl)aminoalkyl; alkoxycarbonylalkyl; and alkylcarboxyalkyl.

In some embodiments, R₃, R₇, and R₁₂ are independently selected from the group consisting of aminoalkyloxy and aminoalkylcarboxy, and wherein R₁₈ is selected from the group consisting of alkylaminoalkyl; di-(alkyl)aminoalkyl; and alkoxycarbonylalkyl.

In some embodiments, R₃, R₇, R₁₂, and R₁₈ are independently selected from the group consisting of amino-C₃-alkyloxy; amino-C₃-alkyl-carboxy; C₈-alkylamino-C₅-alkyl; C₁₂-alkylamino-C₅-alkyl; C₁₃-alkylamino-C₅-alkyl; C₁₆-alkylamino-C₅-alkyl; di-(C₅-alkyl)amino-C₅-alkyl; C₆-alkoxy-carbonyl-C₄-alkyl; C₈-alkoxy-carbonyl-C₄-alkyl; C₁₀-alkoxy-carbonyl-C₄-alkyl; C₆-alkyl-carboxy-C₄-alkyl; C₈-alkyl-carboxy-C₄-alkyl; and C₁₀-alkyl-carboxy-C₄-alkyl.

In some embodiments, R₃, R₇, R₁₂, and R₁₈ are independently selected from the group consisting of amino-C₃-alkyloxy; amino-C₃-alkyl-carboxy; C₈-alkylamino-C₅-alkyl; C₁₂-alkylamino-C₅-alkyl; C₁₃-alkylamino-C₅-alkyl; C₁₆-alkylamino-C₅-alkyl; di-(C₅-alkyl)amino-C₅-alkyl; C₆-alkoxy-carbonyl-C₄-alkyl; C₈-alkoxy-carbonyl-C₄-alkyl; and C₁₀-alkoxy-carbonyl-C₄-alkyl.

In some embodiments, R₃, R₇, and R₁₂, are independently selected from the group consisting of amino-C₃-alkyloxy or amino-C₃-alkyl-carboxy, and wherein R₁₈ is selected from the group consisting of C₈-alkylamino-C₅-alkyl; C₁₂-alkylamino-C₅-alkyl; C₁₃-alkylamino-C₅-alkyl; C₁₆-alkylamino-C₅-alkyl; di-(C₅-alkyl)amino-C₅-alkyl; C₆-alkoxy-carbonyl-C₄-alkyl; C₈-alkoxy-carbonyl-C₄-alkyl; C₁₀-alkoxy-carbonyl-C₄-alkyl; C₆-alkyl-carboxy-C₄-alkyl; C₈-alkyl-carboxy-C₄-alkyl; and C₁₀-alkyl-carboxy-C₄-alkyl.

In some embodiments, R₃, R₇, and R₁₂, are independently selected from the group consisting of amino-C₃-alkyloxy or amino-C₃-alkyl-carboxy, and wherein R₁₈ is selected from the group consisting of C₈-alkylamino-C₅-alkyl; C₁₂-alkylamino-C₅-alkyl; C₁₃-alkylamino-C₅-alkyl; C₁₆-alkylamino-C₅-alkyl; di-(C₅-alkyl)amino-C₅-alkyl; C₆-alkoxy-carbonyl-C₄-alkyl; C₈-alkoxy-carbonyl-C₄-alkyl; and C₁₀-alkoxy-carbonyl-C₄-alkyl.

In some embodiments, R₃, R₇, R₁₂, and R₁₈ are independently selected from the group consisting of amino-C₃-alkyloxy; amino-C₃-alkyl-carboxy; amino-C₂-alkylcarboxy; C₈-alkylamino-C₅-alkyl; C₈-alkoxy-carbonyl-C₄-alkyl; C₁₀-alkoxy-carbonyl-C₄-alkyl; C₈-alkyl-carbonyl-C₄-alkyl; di-(C₅-alkyl)amino-C₅-alkyl; C₁₃-alkylamino-C₅-alkyl; C₆-alkoxy-carbonyl-C₄-alkyl; C₆-alkyl-carboxy-C₄-alkyl; C₁₆-alkylamino-C₅-alkyl; C₁₂-alkylamino-C₅-alkyl; and hydroxy(C₅)alkyl.

In some embodiments, R₁₈ is selected from the group consisting of C₈-alkylamino-C₅-alkyl or C₈-alkoxy-carbonyl-C₄-alkyl.

In some embodiments, at least R₁₈ can have the following structure:

—R₂₀—(C═O)—N—R₂₁R₂₂

wherein R₂₀ is omitted or alkyl, alkenyl, alkynyl, or aryl, and R₂₁ and R₂₂ are independently selected from the group consisting of hydrogen, alkyl, alkenyl, alkynyl, or aryl, provided that at least one of R₂₁ and R₂₂ is not hydrogen.

In some embodiments, R₂₁ and R₂₂ are independently selected from the group consisting of hydrogen, C₁-C₂₄ alkyl, C₂-C₂₄ alkenyl, C₂-C₂₄ alkynyl, C₆ or C₁₀ aryl, 5 to 10 membered heteroaryl, 5 to 10 membered heterocyclyl, C₇-C₁₃ aralkyl, (5 to 10 membered heteroaryl)-C₁-C₆ alkyl, C₃-C₁₀ carbocyclyl, C₄-C₁₀ (carbocyclyl)alkyl, (5 to 10 membered heterocyclyl)-C₁-C₆ alkyl, amido, and a suitable amine protecting group, provided that at least one of R₂₁ and R₂₂ is not hydrogen. In some embodiments, R₂₁ and R₂₂, together with the atoms to which they are attached, form a 5 to 10 membered heterocyclyl ring.

In some embodiments, one or more of rings A, B, C, and D are heterocyclic.

In some embodiments, rings A, B, C, and D are non-heterocyclic.

In some embodiments, the CSA compound is a compound of Formula IV, which is a subset of Formula III, or salt thereof, having a steroidal backbone:

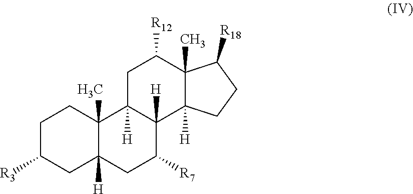

In some embodiments, R₃, R₇, and R₁₂ are independently selected from the group consisting of hydrogen, an unsubstituted (C₁-C₂₂) alkyl, unsubstituted (C₁-C₂₂) hydroxyalkyl, unsubstituted (C₁-C₂₂) alkyloxy-(C₁-C₂₂) alkyl, unsubstituted (C₁-C₂₂) alkylcarboxy-(C₁-C₂₂) alkyl, unsubstituted (C₁-C₂₂) alkylamino-(C₁-C₂₂)alkyl, unsubstituted (C₁-C₂₂) alkylamino-(C₁-C₂₂) alkylamino, unsubstituted (C₁-C₂₂) alkylamino-(C₁-C₂₂) alkylamino-(C₁-C₁₈) alkylamino, an unsubstituted (C₁-C₂₂) aminoalkyl, an unsubstituted arylamino-(C₁-C₂₂) alkyl, an unsubstituted (C₁-C₂₂) aminoalkyloxy, an unsubstituted (C₁-C₂₂) aminoalkyloxy-(C₁-C₂₂) alkyl, an unsubstituted (C₁-C₂₂) aminoalkylcarboxy, an unsubstituted (C₁-C₂₂) aminoalkyl-aminocarbonyl, an unsubstituted (C₁-C₂₂) aminoalkylcarboxamido, an unsubstituted di(C₁-C₂₂ alkyl)aminoalkyl, unsubstituted (C₁-C₂₂) guanidinoalkyloxy, unsubstituted (C₁-C₂₂) quaternary ammonium alkylcarboxy, and unsubstituted (C₁-C₂₂) guanidinoalkyl carboxy.

In some embodiments, R₃, R₇, and R₁₂ are independently selected from the group consisting of hydrogen, an unsubstituted (C₁-C₆) alkyl, unsubstituted (C₁-C₆) hydroxyalkyl, unsubstituted (C₁-C₁₆) alkyloxy-(C₁-C₅) alkyl, unsubstituted (C₁-C₁₆) alkylcarboxy-(C₁-C₅) alkyl, unsubstituted (C₁-C₁₆) alkylamino-(C₁-C₅)alkyl, unsubstituted (C₁-C₁₆) alkylamino-(C₁-C₅) alkylamino, unsubstituted (C₁-C₁₆) alkylamino-(C₁-C₁₆) alkylamino-(C₁-C₅) alkylamino, an unsubstituted (C₁-C₁₆) aminoalkyl, an unsubstituted arylamino-(C₁-C₅) alkyl, an unsubstituted (C₁-C₅) aminoalkyloxy, an unsubstituted (C₁-C₁₆) aminoalkyloxy-(C₁-C₅) alkyl, an unsubstituted (C₁-C₅) aminoalkylcarboxy, an unsubstituted (C₁-C₅) aminoalkylaminocarbonyl, an unsubstituted (C₁-C₅) aminoalkylcarboxamido, an unsubstituted di(C₁-C₅ alkyl)amino-(C₁-C₅) alkyl, unsubstituted (C₁-C₅) guanidinoalkyloxy, unsubstituted (C₁-C₁₆) quaternary ammonium alkylcarboxy, and unsubstituted (C₁-C₁₆) guanidinoalkylcarboxy.

In some embodiments, R₃, R₇, and R₁₂ are independently selected from the group consisting of aminoalkyloxy; aminoalkylcarboxy; alkylaminoalkyl; alkoxycarbonylalkyl; alkylcarbonylalkyl; di(alkyl)aminoalkyl; alkylcarboxyalkyl; and hydroxyalkyl.

In some embodiments, R₃, R₇, and R₁₂ are independently selected from the group consisting of aminoalkyloxy and aminoalkylcarboxy.

In some embodiments, R₃, R₇, and R₁₂ are the same. In some embodiments, R₃, R₇, and R₁₂ are aminoalkyloxy. In some embodiments, R₃, R₇, and R₁₂ are aminoalkylcarboxy.

In some embodiments, R₃, R₇, and R₁₂ are independently selected from the group consisting of amino-C₃-alkyloxy; amino-C₃-alkyl-carboxy; C₈-alkylamino-C₅-alkyl; C₈-alkoxy-carbonyl-C₄-alkyl; C₈-alkyl-carbonyl-C₄-alkyl; di-(C₅-alkyl)amino-C₅-alkyl; C₁₃-alkylamino-C₅-alkyl; C₆-alkoxy-carbonyl-C₄-alkyl; C₆-alkyl-carboxy-C₄-alkyl; and C₁₆-alkylamino-C₅-alkyl.

In some embodiments, CSA compounds as disclosed herein can be a compound of Formula I, Formula II, Formula III, Formula IV, or salts thereof wherein at least R₁₈ of the steroidal backbone includes amide functionality in which the carbonyl group of the amide is positioned between the amido nitrogen of the amide and fused ring D of the steroidal backbone. For example, any of the embodiments described above can substitute R₁₈ for an R₁₈ including amide functionality in which the carbonyl group of the amide is positioned between the amido nitrogen of the amide and fused ring D of the steroidal backbone.

In some embodiments, one or more of R₃, R₇, or R₁₂ may include a guanidine group as a cationic functional group and may be bonded to the steroid backbone by an ether linkage. For example, one or more of R₃, R₇, or R₁₂ may be a guanidinoalkyloxy group. An example includes H₂N—C(═NH)—NH-alkyl-O—,

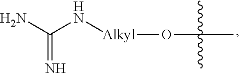

wherein the alkyl portion is defined as with the embodiments described above. In a preferred embodiment, the alkyl portion is a straight chain with 3 carbon atoms, and therefore one or more of R₃, R₇, or R₁₂ may be a guanidinopropyloxy group.

One of skill in the art will recognize that other cationic functional groups may be utilized, and that the cationic functional groups may be bonded to the steroid backbone through a variety of other tethers or linkages. For example, the cationic functional groups may be bonded to the steroid backbone by an ester linkage. For example, one or more of R₃, R₇, or R₁₂ may be an aminoalkylcarboxy or guanidinoalkylcarboxy, such as H₂N-alkyl-C(═O)—O— or H₂N—C(═NH)—NH-alkyl-C(═O)—O—, wherein the alkyl portion is defined as with the embodiments described above. In other embodiments, the cationic functional groups may be bonded to the steroid backbone by an amide linkage. For example, one or more of R₃, R₇, or R₁₂ may be an aminoalkylcarbonylamino (i.e. aminoalkylcarboxamido) or guanidinoalkylcarbonylamino (i.e. guanidinoalkylcarboxamido), such as H₂N-alkyl-C(═O)—NH— or H₂N—C(═NH)—NH-alkyl-C(═O)—NH—, wherein the alkyl portion is defined as with the embodiments described above.

Additionally, one of skill in the art will recognize that the tethers may be of varying lengths. For example, the length between the steroid backbone and the cationic functional group (e.g., amino or guanidino group), may be between 1 and 15 atoms or even more than 15 atoms. In other embodiments, the length may be between 1 and 8 atoms. In a preferred embodiment, the length of the tether is between two and four atoms. In other embodiments, there is no tether, such that the cationic functional group is bonded directly to the steroid backbone.

One of skill in the art will also note that the various cationic functional groups of the present disclosure may be utilized in combination, such that one or more of R₃, R₇, or R₁₂ may include one variation of cationic functional group while one or more of another of R₃, R₇, or R₁₂ of the same compound may include a different variation of cationic functional group. Alternatively, two or more of R₃, R₇, or R₁₂ may include the same cationic functional group, or all of R₃, R₇, or R₁₂ may include the same cationic functional group (in embodiments where all of R₃, R₇, or R₁₂ are cationic functional groups).

Additionally, although in a preferred embodiment one or more cationic functional groups are disposed at R₃, R₇, or R₁₂, one of skill in the art will recognize that in other embodiments, R₃, R₇, or R₁₂ may not be cationic functional groups and/or one or more cationic functional groups may be disposed at other locations of the steroid backbone. For example, one or more cationic functional groups may be disposed at R₁, R₂, R₃, R₄, R₆, R₇, R₁₁, R₁₂, R₁₅, R₁₆, R₁₇, and/or R₁₈.

The compounds and compositions disclosed herein are optionally prepared as salts. The term “salt” as used herein is a broad term, and is to be given its ordinary and customary meaning to a skilled artisan (and is not to be limited to a special or customized meaning), and refers without limitation to a salt of a compound. In some embodiments, the salt is an acid addition salt of the compound. Salts can be obtained by reacting a compound with inorganic acids such as hydrohalic acid (e.g., hydrochloric acid or hydrobromic acid), sulfuric acid, nitric acid, and phosphoric acid. Salts can also be obtained by reacting a compound with an organic acid such as aliphatic or aromatic carboxylic or sulfonic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of preventing microbial fouling of a medical implant, comprising:
providing a medical implant having a plurality of incorporated cationic steroidal antimicrobial (CSA) molecules;
implanting the medical implant, and
the medical implant with incorporated CSA molecules killing microbes contacting the medical implant or preventing adherence of microbes contacting the medical implant to thereby prevent microbial colonization and fouling of the medical implant.

2. The method of claim 1, wherein the medical implant is selected from the group consisting of catheters, vascular catheters, peritoneal dialysis catheters, urinary catheters, joint prostheses, penile implants, dialysis access devices, dialysis access grafts, hemodialysis devices, fistula devices, hemodialysis grafts, cardiac devices, prosthetic valves, pacemakers (including implantable cardioverter defibrillators, or ICDs, and vascular assist devices, or VADs), central nervous system devices (VPSs), endotracheal tubes, intravenous (IV) needles, IV feed lines, other IV components, feeder tubes, drains, prosthesis components (e.g., voice prostheses), peristaltic pumps, tympanostomy tubes, tracheostomy tubes, oral care devices (dentures, dental implants), intrauterine devices (IUDs), cardiac implants, and dermal fillers.

3. The method of claim 1, wherein the medical implant includes silicone.

4. The method of claim 1, wherein the medical implant comprises a coating, the coating including CSA molecules incorporated therein.

5. The method of claim 4, wherein the coating is a hydrogel.

6. The method of claim 1, wherein the CSA molecules include CSA-131 or a salt thereof.

7. The method of claim 6, wherein the CSA molecules include an NDSA salt of CSA-131.

8. The method of claim 1, wherein the CSA molecules include one or more sulfonic acid addition salts.

9. The method of claim 8, wherein the one or more sulfonic acid addition salts includes a 1,5-naphthalenedisulfonic acid salt.

10. The method of claim 1, wherein the method reduces or prevents fouling caused by a biofilm.

11. The method of claim 10, wherein the biofilm is a bacterial and/or fungal biofilm.

12. The method of claim 1, wherein the method prevents fouling caused by Candida spp.

13. The method of claim 1, wherein microbial fouling is reduced, as compared to use of a medical implant not incorporating the CSA molecules, by a Log₁₀ reduction of greater than about 4, or greater than about 6, or about 4 to about 10.

14. The method of claim 1, wherein the microbial fouling is prevented for about 7 days or more, or 14 days or more, or one month or more, or three months or more following implantation.

15. The method of claim 1, wherein the medical implant is at least partially formed from a polymeric material, and wherein the CSA molecules are distributed throughout the polymeric material.

16. A method of preventing microbial fouling of a medical implant, comprising:
placing a medical implant having a plurality of incorporated cationic steroidal antimicrobial (CSA) molecules into an environment prone to microbial colonization and fouling of medical implants; and
the medical implant with incorporated CSA molecules killing microbes contacting the medical implant or preventing adherence of microbes contacting the medical implant to thereby prevent microbial colonization and fouling of the medical implant.

17. A method of manufacturing a medical implant capable of reducing or preventing microbial fouling, the method comprising:
mixing a plurality of cationic steroidal antimicrobial (CSA) molecules with a biologically compatible moldable polymeric material; and
forming the moldable polymeric material into a medical implant.

18. The method of claim 17, wherein the medical implant is selected from the group consisting of catheters, vascular catheters, peritoneal dialysis catheters, urinary catheters, joint prostheses, penile implants, dialysis access devices, dialysis access grafts, hemodialysis devices, fistula devices, hemodialysis grafts, cardiac devices, prosthetic valves, pacemakers (including implantable cardioverter defibrillators, or ICDs, and vascular assist devices, or VADs), central nervous system devices (VPSs), endotracheal tubes, intravenous (IV) needles, IV feed lines, other IV components, feeder tubes, drains, prosthesis components (e.g., voice prostheses), peristaltic pumps, tympanostomy tubes, tracheostomy tubes, oral care devices (dentures, dental implants), intrauterine devices (IUDs), cardiac implants, and dermal fillers.

19. The method of claim 17, wherein the polymeric material is formed into the medical implant by extrusion.

20. The method of claim 17, wherein the polymeric material includes silicone.

21. The method of claim 17, wherein the CSA molecules include a naphthalenedisulfonic acid (NDSA) salt of a CSA compound, such as CSA-131.

22. A method of manufacturing a medical implant capable of reducing or preventing microbial fouling, the method comprising:
forming a biologically compatible moldable polymeric material into a medical implant;
mixing a plurality of CSA molecules with a coating material to form a CSA coating; and
applying to the CSA coating to a surface of the medical implant.

23. The method of claim 23, wherein the medical implant is selected from the group consisting of catheters, vascular catheters, peritoneal dialysis catheters, urinary catheters, joint prostheses, penile implants, dialysis access devices, dialysis access grafts, hemodialysis devices, fistula devices, hemodialysis grafts, cardiac devices, prosthetic valves, pacemakers (including implantable cardioverter defibrillators, or ICDs, and vascular assist devices, or VADs), central nervous system devices (VPSs), endotracheal tubes, intravenous (IV) needles, IV feed lines, other IV components, feeder tubes, drains, prosthesis components (e.g., voice prostheses), peristaltic pumps, tympanostomy tubes, tracheostomy tubes, oral care devices (dentures, dental implants), intrauterine devices (IUDs), cardiac implants, and dermal fillers.
